(19) United States
(12) Patent Application Publication
(10) Pub. No.: US 2024/0003891 A1
(43) Pub. Date: Jan. 4, 2024

(54) IMMUNOASSAY USING CARBON NANOMATERIALS AND METHOD OF DETECTING TARGET ANTIGEN USING THE SAME

(71) Applicant: Ji Hoon LEE, Gaithersburg, MD (US)

(72) Inventor: Ji Hoon LEE, Gaithersburg, MD (US)

(21) Appl. No.: 18/124,986

(22) Filed: Mar. 22, 2023

Related U.S. Application Data
(60) Provisional application No. 63/357,892, filed on Jul. 1, 2022.

(51) Int. Cl. G01N 33/58; C07K 7/08

(52) U.S. Cl. CPC G01N 33/582; C07K 2317/55; C07K 7/08

(57) ABSTRACT

The disclosed is a device and a method for detecting a target antigen in a sample, the device includes: a container; a carbon nanomaterial; and an antibody conjugated with a marker, where the antibody is immobilized on a surface of the carbon nanomaterial, where the antibody has a binding site of the target antigen, and where, when the target antigen binds to the binding site of the antibody, the antibody conjugated with the marker detaches from the carbon nanomaterial and the marker-labeled antibody bound with the target antigen generates a signal.

### CROSS-REFERENCE TO RELATED APPLICATION

This application claims priority to and the benefit of U.S. Provisional Application No. 63/357,892, filed on Jul. 1, 2022 in the United States Patent & Trademark office, the entire content of which is incorporated herein by reference.

### TECHNICAL FIELD

The present disclosure relates to the field of immunoassay techniques for detecting and quantifying target materials in biological and/or chemical specimens, and in particular to a novel device and a method for detecting a target antigen using a carbon nanomaterial on which a labeled antibody is immobilized on its surface.

### BACKGROUND

Immunoassay methods are widely applied to early diagnose and accurately prognose human diseases. Additionally, viruses, bacteria, and toxins are detected and monitored using the immunoassays. Currently, immunoassays can be divided into two different methods, which are sandwich immunoassay and competitive immunoassay.

Sandwich immunoassays, operated with a capture antibody and a detection antibody, have been used to quantify large biomolecules (or antigens), such as virus, cell, protein, and peptide. In order to enhance the sensitivity of the sandwich immunoassays, a detection antibody is conjugated with a specific material such as enzyme (e.g., horseradish peroxidase (HRP), alkaline phosphatase (AP)), fluorescent dye, radio isotope, gold nanoparticle, single- or double-stranded DNA, carbon dots, and quantum dots. In order to enhance the sensitivity, accuracy, and reliability of the sandwich immunoassay, multiple washing procedures are necessary. With the increase of antigen concentration in a sample, signal generated from the detection antibody conjugated with a labeling material are proportionally enhanced. In conclusion, a specific antigen in a sample can be quantified through the time-consuming multiple steps to operate the sandwich immunoassay.

Competitive immunoassay is operated with a single antibody and a constant amount of an antigen conjugated with a labeling material which is the same as that bound with a detection antibody applied in sandwich immunoassay. The competitive immunoassays are applied to quantify and monitor small molecules (or antigens), such as hormones and chemicals. Multiple washing procedures are also necessary to accurately and reliably quantify antigens in a sample. The antigens in a sample and the labelled antigen competitively bind with the single antibody coated on the solid surface. Thus, with the increase of antigen concentration in a sample, signal generated from the labelled antigen is proportionally decreased. In conclusion, it is difficult to quantify trace levels of antigen because the signal measured in the absence of antigen is too high to detect the low concentration of antigen. The sensitivity of the competitive immunoassay is not as good as that of the sandwich immunoassay.

The background description provided herein is for the purpose of generally presenting context of the disclosure. Unless otherwise indicated herein, the materials described in this section are not prior art to the claims in this application and are not admitted to be prior art, or suggestions of the prior art, by inclusion in this section.

### SUMMARY

An object of the present disclosure is to provide a device for detecting a target antigen in a sample, a method of detecting a target antigen and a method of quantifying a target antigen based on a novel immunoassay method using carbon nanomaterials and dual roles of an antibody conjugated with a marker capable of capturing and detecting a specific antigen in a sample

The novel immunoassay according to the present disclosure is hereinafter referred to as ‘all-in-one immunoassay’ for the sole purpose of distinguishing it from the conventional immunoassay methods, and thus, this term “all-in-one” does not limit the scope of the invention disclosed herein. In the all-in-one immunoassay, an antibody conjugated with a marker rapidly reacts with a specific antigen and generate signal without time consuming procedures such as multiple immunoreactions and washings.

According to an exemplary embodiment, the present disclosure provides a device for detecting a target antigen in a sample, comprising: a container; a carbon nanomaterial; and an antibody conjugated with a marker, wherein the antibody is immobilized on a surface of the carbon nanomaterial, wherein the antibody has a binding site of the target antigen, and wherein, when the target antigen binds to the binding site of the antibody, the antibody conjugated with the marker detaches from the carbon nanomaterial and the marker-labeled antibody bound with an antigen (marker<antibody>antigen) generates a signal. The signal may be proportionally enhanced with the increase of the antigens in a sample.

The carbon nanomaterial may be one or more selected from the group consisting of graphene, graphene oxide, reduced graphene oxide, carbon nanotubes, and carbon nanotube oxides.

The antibody may be immobilized on the surface of the carbon nanomaterial by a non-covalent 7C-7C stacking interaction, and a distance between the antibody and the carbon nanomaterial is 10 nm or less.

The sample may be a biological solution selected from the group consisting of serum, plasma, whole blood, sweat, urine, and cerebrospinal fluid.

The signal emitted from the marker is selected from the group consisting of bioluminescence, chemiluminescence, fluorescence, colorimetric, electrochemical, electrochemiluminescence, radiometric, and light visible to the naked eye.

The marker may be a bioluminescence marker selected from the group consisting of luciferase, luciferin, and luciferin derivatives.

The marker may be a chemiluminescence detection marker of a detection selected from the group consisting of acridinium ester chemiluminescence detection, chemiluminescence using horseradish peroxidase (HRP)-labeled antibody, chemiluminescence detection using alkaline phosphatase (ALP)-labeled antibody, phenylglyoxal derivative chemiluminescence detection, and ODI chemiluminescence detection operated with an antibody conjugated with luminescent dyes.

The marker may be a colorimetric detection marker of a detection selected from the group consisting of colorimetric detection operated with antigen-bound antibody conjugated with HRP, colorimetric detection operated with antigen-bound antibody conjugated with ALP, colorimetric detection operated with antigen-bound antibody conjugated with β-galactosidase, and colorimetric detection operated with antigen-bound antibody conjugated with glucose oxidase.

The marker may be an electrochemiluminescence detection marker selected from the group consisting of an antibody labeled with a ruthenium complex (Ru(bpy)₃²⁺), a nanoparticle (e.g., gold, platinum, silver) or N-(4-aminobutyl)-N-ethylisoluminol (ABEI) and a specific substrate (e.g., tripropylamine, H₂O₂), and an electrode.

The marker may be an electrochemical detection marker of a detection selected from the group consisting of an antibody labeled with an enzyme (e.g., HRP, ALP, glucose oxidase, β-galactosidase), a specific substrate, and an electrode.

The marker may be a fluorescence detection marker of a detection selected from the group consisting of (1) an enzyme (e.g., HRP.ALP)-labeled antibody and specific substrates (e.g., Amplex Red, H₂O₂, fluorescein diphosphate (FDP), 4-methylumbelliferyl phosphate) and (2) a fluorescence dye-labeled antibody.

The marker may be a naked-eye detection marker of a detection selected from the group consisting of an enzyme (e.g., HRP, ALP) and a specific substrate (e.g., Amplex Red, Zonyl FSN-100 functionalized gold nanoparticles) capable of developing a color.

The marker may be a radioactive detection marker of a detection selected from the group consisting of an isotope (e.g., ¹²⁵I)-labeled antibody.

According to another exemplary embodiment, the present disclosure provides a method of manufacturing the above device, comprising: fixing the carbon nanomaterial on an inner surface of the container; providing the antibody conjugated with the marker; and bringing the antibody conjugated with the marker into contact with a surface of the carbon nanomaterial.

The carbon nanomaterial may be a magnetic graphene, graphene oxide and reduced graphene oxide, single-walled carbon nanotube, and multi-walled carbon nanotube.

The carbon nanomaterial may have a form of a thin film, composed of graphene oxide, and the container is a polystyrene container.

According to another exemplary embodiment, the present disclosure provides a method of detecting a target antigen, comprising: introducing a sample solution to the above device; and detecting a signal generated from the marker-labeled antibody bound with the target antigen (marker<antibody>antigen) to detect the target antigen in the sample solution.

The carbon nanomaterial may be one or more selected from the group consisting of graphene, graphene oxide, reduced graphene oxide, single-walled carbon nanotubes, and multi-walled carbon nanotubes.

The carbon nanomaterial has a form of a thin film composed of graphene oxide, and the container is a polystyrene container.

The antibody may be immobilized on the surface of the carbon nanomaterial by a non-covalent 7C-7C stacking interaction, and a distance between the antibody and the carbon nanomaterial is 10 nm or less.

The sample solution may be a biological solution selected from the group consisting of serum, plasma, whole blood, sweat, urine, and cerebrospinal fluid.

The signal generated from the marker-labeled antibody bound with the target antigen is selected from the group consisting of bioluminescence, chemiluminescence, fluorescence, colorimetric, electrochemical, electrochemiluminescence, radiometric, and light visible to the naked eye.

The method may not use an artificially manufactured antigen conjugated with the marker.

The method may not use a detection antibody conjugated with a marker.

The method may comprise two or more types of antibodies.

The method may not comprise a washing procedure to remove waste after introducing the sample solution to the device.

The signal may be emitted within 30 minutes after introducing the sample solution containing the target antigen to the device.

According to another exemplary embodiment, the present disclosure provides a method of quantifying a target antigen, comprising: introducing a sample solution to the above device; and measuring an intensity of the signal to quantify the target antigen in the sample solution.

The carbon nanomaterial may be one or more selected from the group consisting of graphene, graphene oxide, reduced graphene oxide, single-walled carbon nanotubes, and multi-walled carbon nanotubes.

The antibody may be immobilized on the surface of the carbon nanomaterial by a non-covalent 7C-7C stacking interaction, and a distance between the antibody and the carbon nanomaterial is 10 nm or less.

The sample solution may be a biological solution selected from the group consisting of serum, plasma, whole blood, sweat, urine, and cerebrospinal fluid.

The signal emitted from the marker is selected from the group consisting of bioluminescence, chemiluminescence, fluorescence, colorimetric, electrochemical, electrochemiluminescence, radiometric, and light visible to the naked eye.

The method may not use an artificially manufactured antigen conjugated with the marker.

The method may not use a detection antibody conjugated with the marker.

The method may comprise two or more types of antibodies.

The method may not comprise a washing procedure to remove waste after introducing the sample solution to the device. The intensity of the signal may proportionally increase when a concentration of the target antigen in the sample solution increases.

The signal may be emitted within 30 minutes after introducing the sample solution to the device.

### BRIEF DESCRIPTION OF THE DRAWINGS

The accompanying drawings illustrate a preferred embodiment of the present disclosure and together with the foregoing disclosure, serve to provide further understanding of the technical features of the present disclosure, and thus, the present disclosure is not construed as being limited to the drawing.

FIG. 1A shows a structure of an antibody and FIG. 1B shows an interaction between antibodies and carbon nanomaterials in the absence and presence of an antigen capable of strongly binding the antibody according to one embodiment of the present disclosure.

FIG. 2 shows schematic view of the all-in-one immunoassay with chemiluminescence detection according to another embodiment of the present disclosure.

FIG. 3 shows a conventional sandwich immunoassay with chemiluminescence detection.

FIG. 4 shows a conventional competitive immunoassay with chemiluminescence detection.

FIG. 5 is a graph showing that the all-in-one immunoassay with ODI chemiluminescence detection for the quantification of T4 in human serum according to another embodiment of the present disclosure.

FIG. 6 is a graph showing that the all-in-one immunoassay with acridinium ester chemiluminescence detection for the quantification of CA-19 in human serum according to another embodiment of the present disclosure.

FIG. 7 shows a schematic view of bioluminescence detection using luciferase labeled antibody in the all-in-one immunoassay according to another embodiment of the present disclosure.

FIG. 8A shows bioluminescence detection using 6-amino-D-luciferin and FIG. 8B shows bioluminescence detection using 6-aminoseleno-D-luciferin labeled antibody-bound an antigen according to another embodiment of the present disclosure.

FIG. 9 shows acridinium NHS ester derivatives that can be used in the all-in-one immunoassay according to another embodiment of the present disclosure.

FIG. 10A shows acridinium ester chemiluminescence reaction mechanism using acridinium C2, NHS ester and FIG. 10B shows acridinium ester chemiluminescence reaction mechanism using Lumiwo™ acridinium NHS ester labeled antibody in the all-in-one immunoassay according to another embodiment of the present disclosure.

FIG. 11A shows the all-in-one immunoassay using acridinium ester linked with fluorescein, FIG. 11B shows the all-in-one immunoassay using acridinium ester linked with rhodamine B, and FIG. 11C shows the all-in-one immunoassay using acridinium ester linked with BDP according to another embodiment of the present disclosure.

FIG. 12 shows the all-in-one immunoassay with luminol chemiluminescence detection using an antibody conjugated with HRP according to another embodiment of the present disclosure.

FIG. 13 shows the all-in-one immunoassay with luminol chemiluminescence detection using an antibody conjugated with ABEI according to another embodiment of the present disclosure.

FIG. 14 shows the all-in-one immunoassay with ODI chemiluminescence detection using an antibody conjugated with HRP in the presence of Amplex Red. 1: Amplex Red, 2: resorufin under the ground state, 3: resorufin under the excited state, 4: ODI, 5: high-energy intermediate formed from the reaction of ODI and H₂O₂ according to another embodiment of the present disclosure.

FIG. 15 shows the all-in-one immunoassay with chemiluminescence detection operated based on the reaction of 3-(2′-spiroadamantyl)-4-methoxy-4-(3″-phosphoryloxy)-phenyl-1,2-dioxetane (Adamantyl 1,2-dioxetane aryl phosphate, AMPPD), which is one of adamantyl 1,2-dioxetane phosphate derivatives, and ALP according to another embodiment of the present disclosure.

FIG. 16 shows the all-in-one immunoassay with ODI chemiluminescence detection using ALP enzyme reaction in the presence of FDP. 1: FDP, 2: fluorescein under the ground state, 3: fluorescein under the excited state, 4: ODI, X: high-energy intermediate formed from the reaction of ODI and H₂O₂ according to another embodiment of the present disclosure.

FIG. 17 shows the all-in-one immunoassay with ODI chemiluminescence detection using ALP enzyme reaction in the presence of 4-MUP. 1: 4-MUP, 2: 4-MU under the ground state, 3: 4-MU under the excited state, 4: ODI, X: high-energy intermediate formed from the reaction of ODI and H₂O₂ according to another embodiment of the present disclosure.

FIG. 18 shows the target antigen bound with an antibody conjugated with single stranded DNA (20-100 bp) and a luminescent dye. 1: Pacific Blue, 2: 6-Carboxyfluorescein (6-FAM), 3. Fluorescein according to another embodiment of the present disclosure.

FIG. 19 shows the all-in-one immunoassay with phenylglyoxal derivative chemiluminescence detection. 1. Cytosine, 2: Adenine, 3: Guanine, 4: Thymine, 5. Pacific Blue under the ground state, 6: High-energy intermediate formed from the reaction of guanine and TMPG (1-10 mM) in the presence of TPA (15-30 mM) and DMF, 7: Pacific Blue under the excited state according to another embodiment of the present disclosure.

FIG. 20 shows the multiplex all-in-one immunoassay with phenylglyoxal derivative chemiluminescence detection capable of simultaneously quantifying three different target antigens in a sample. 1: Pacific Blue, 2: 6-FAM, 3: Rhodamine 101 (or ROX) according to another embodiment of the present disclosure.

FIG. 21A shows the all-in-one immunoassay with ODI chemiluminescence detection operated with an antibody conjugated with rhodamine 101 through a linker and FIG. 21B shows the all-in-one immunoassay with ODI chemiluminescence detection operated with an antibody conjugated with rhodamine 101 through a single stranded DNA according to another embodiment of the present disclosure.

FIG. 22 shows the multiplex all-in-one immunoassay with ODI chemiluminescence detection capable of simultaneously quantifying three different target antigens in a sample. 1: Pacific Blue, 2: 6-FAM, 3: Rhodamine 101 (or ROX) according to another embodiment of the present disclosure.

FIG. 23 shows the all-in-one immunoassay with a colorimetric detection using a HRP labeled antibody capable of capturing a target antigen and TMB (3,3′, 5,5′-tetramethylbenzidine) as a substrate according to another embodiment of the present disclosure.

FIG. 24 shows the all-in-one immunoassay with a colorimetric detection using a HRP labeled antibody capable of capturing a target antigen and ABTS (2,2′-azino-bis (3-ethylbenzothiazoline-6-sulphonic acid)) as a substrate according to another embodiment of the present disclosure.

FIG. 25 shows the all-in-one immunoassay with a colorimetric detection using a HRP labeled antibody capable of capturing a target antigen and OPD (o-phenylenediamine dihydrochloride) as a substrate according to another embodiment of the present disclosure.

FIG. 26 shows the all-in-one immunoassay with a colorimetric detection using a HRP labeled antibody capable of capturing a target antigen and Amplex Red as a substrate according to another embodiment of the present disclosure.

FIG. 27 shows the all-in-one immunoassay with a colorimetric detection using an ALP labeled antibody capable of capturing a target antigen and pNPP (p-Nitrophenyl Phosphate, Disodium Salt) substrate according to another embodiment of the present disclosure.

FIG. 28 shows the all-in-one immunoassay with a colorimetric detection using a β-galactosidase labeled antibody capable of capturing a target antigen and ONPG (o-nitrophenyl-β-D-galactopyranoside) as a substrate according to another embodiment of the present disclosure.

FIG. 29 shows the all-in-one immunoassay with a colorimetric detection using a glucose oxidase labeled antibody capable of capturing a target antigen and TMB as a substrate according to another embodiment of the present disclosure.

FIG. 30 shows the all-in-one immunoassay with an electrochemical detection using an ALP labeled antibody capable of capturing a target antigen and 3-indoxyl phosphate disodium salt as a substrate. 1: 3-indoxyl phosphate disodium salt, 2: Indigo blue according to another embodiment of the present disclosure.

FIG. 31 shows the all-in-one immunoassay with an electrochemical detection using an HRP labeled antibody capable of capturing a target antigen, H₂O₂, and acetaminophen as a substrate. 1: Acetaminophen, 2: N-acretyl-4-quinoneimine according to another embodiment of the present disclosure.

FIG. 32 shows the all-in-one immunoassay with an electrochemiluminescence detection operated with a ruthenium complex labeled antibody-bound a target antigen according to another embodiment of the present disclosure.

FIG. 33 shows the all-in-one immunoassay with an electrochemiluminescence detection operated with a gold nanoparticle (Au) labeled antibody-bound a target antigen according to another embodiment of the present disclosure.

FIG. 34 shows the all-in-one immunoassay with an electrochemiluminescence detection operated with a ABEI labeled antibody-bound a target antigen according to another embodiment of the present disclosure.

FIGS. 35A and 35B show the all-in-one immunoassay with a fluorescence detection operated with enzyme-labeled antibodies and a substrate according to another embodiment of the present disclosure.

FIGS. 36A. 36B and 36C show the all-in-one immunoassay with a fluorescence detection operated with fluorescence dye-labeled antibodies according to another embodiment of the present disclosure.

FIG. 37 shows the all-in-one immunoassay capable of sensing positive and negative samples with naked eyes according to another embodiment of the present disclosure.

FIG. 38 shows the all-in-one immunoassay with a radiometer capable of sensing radioactivity according to another embodiment of the present disclosure.

FIG. 39A shows an image of pure graphene oxide observed with a transmission electron microscope (TEM), FIGS. 39B and 39C show images of magnetic graphene oxide obtained from a TEM, FIG. 39D shows separation of magnetic graphene oxide using a magnetic separator according to another embodiment of the present disclosure.

FIG. 40 shows the separation of antibody-conjugated acridinium ester temporarily immobilized on magnetic carbon nanomaterial. Left to right: graphene oxide, reduced graphene oxide, graphene, 10-20 nm multiwalled carbon nanotube, 20-30 nm multiwalled carbon nanotube, and 30-50 nm multiwalled carbon nanotube according to another embodiment of the present disclosure.

FIG. 41 shows CL/CL₀ determined with the all-in-one immunoassays operated with 6 different magnetic carbon nanomaterials according to another embodiment of the present disclosure.

FIG. 42A shows graphene oxide film immobilized on the surface of polystyrene well,

FIG. 42B shows reduced graphene oxide and graphene films on the surface of polystyrene well,

FIG. 42C shows three different sizes of wet and dried multi-walled carbon nanotubes on the polystyrene well according to another embodiment of the present disclosure.

FIG. 43 shows the formation of graphene oxide film on the surface of polystyrene according to another embodiment of the present disclosure. From left to right: Tris-HCl (pH 7), Tris-HCl (pH 7.5), Tris-HCl (pH 8), Tris-HCl (pH 8.5), Tris-HCl (pH 9), TBS (pH 7.4), PBS (7.4), and Sodium phosphate (pH 7).

FIG. 44 shows CL/CL₀ in all-in-one immunoassays operated with graphene oxide films formed in various buffer solutions for the quantification of D-dimer in human serum according to another embodiment of the present disclosure.

FIG. 45 shows CL/CL₀ in all-in-one immunoassays operated with graphene oxide films formed under various pHs in sodium phosphate buffer for the quantification of D-dimer in human serum according to another embodiment of the present disclosure.

FIG. 46 shows CL/CL₀ in all-in-one immunoassays operated with the antibody-conjugated with acridinium ester molecules in various buffers for the quantification of D-dimer in human serum according to another embodiment of the present disclosure.

FIG. 47 shows CL/CL₀ determined with the all-in-one immunoassay with various detections such as bioluminescence, chemiluminescence, and electrochemiluminescence according to another embodiment of the present disclosure.

FIG. 48A shows correlation between acridinium and luminol chemiluminescence used as a detection method of the all-in-one immunoassay, FIG. 48B shows correlation between acridinium and AMPPD chemiluminescence used as a detection method of the all-in-one immunoassay, and FIG. 48C shows correlation between luminol and AMPPD chemiluminescence used as a detection method of the all-in-one immunoassay according to another embodiment of the present disclosure.

### DETAILED DESCRIPTION

While the present disclosure is open to various modifications and alternative embodiments, specific embodiments thereof will be described and illustrated by way of example in the accompanying drawings. However, this is not purported to limit the present disclosure to a specific disclosed form, but it shall be understood to include all modifications, equivalents and substitutes within the idea and the technological scope of the present disclosure.

In this application, it should be understood that terms such as “include” or “have” are intended to indicate that there is a feature, number, step, operation, component, part, or a combination thereof described on the specification, and they do not exclude in advance the possibility of the presence or addition of one or more other features or numbers, steps, operations, components, parts or combinations thereof.

Hereinafter, the present disclosure will be described in detail.

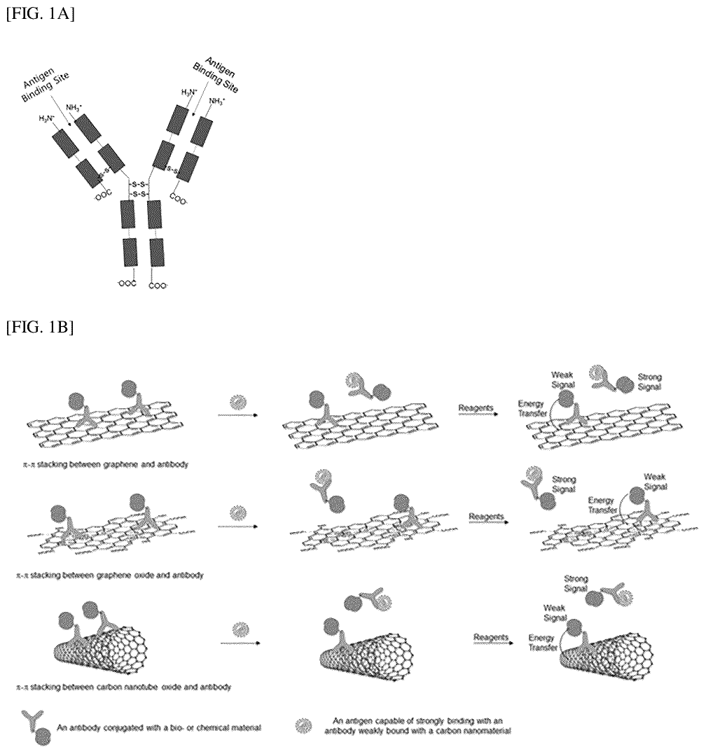

All types of antibodies can weakly but stably bind with carbon nanomaterials with the non-covalent π-π stacking interaction in biological solution such as serum, plasma, whole blood, and cerebrospinal fluids as well as various samples related to environment, public health, and food-safety. As shown in FIG. 1A, in an antibody, multiple amine groups are on the antigen binding sites. These amine groups of the antibody can non-covalently interact with carbon nanomaterials. (See S. Shahriari, M. Sastry, S. Panjikar and R. K. Singh Raman, Nanotechnology, science and applications, 2021, 14, 197-220. See also J. G. Woo, L.-H. Tran, S.-H. Jang, C. Lee and T. J. Kang, Bulletin of the Korean Chemical Society, 2015, 36, 2959-2961.).

Thus, the antigen binding sites of antibodies can bind with carbon nanomaterials due to the non-covalent π-π stacking interaction as shown in FIG. 1B. However, the π-π stacking interaction between a specific antibody and carbon nanomaterials in the presence of an antigen is rapidly decomposed for a strong and permanent binding with the antigen in a solution as shown in FIG. 1B. When the antibodies are immobilized on the surface of carbon nanomaterials by the π-π stacking interaction, the distance between the antibody and the carbon nanomaterials is shorter than 10 nm. Thus, most of the signals generated from the markers (i.e., bio- or chemical-material) labeled antibody on the carbon nanomaterial are lost because the carbon nanomaterials absorb the signal. For example, the strength of chemiluminescence (or fluorescence) emitted in this condition is very weak due to the rapid chemiluminescence (or fluorescence) resonance energy transfer from the bio- or chemical material to the carbon nanomaterial. However, the chemiluminescence (or fluorescence) generated from the bio- or chemical-material labeled antibody, detached from the carbon nanomaterial with the binding interaction between the antibody and the antigen, is strong without any interference from the carbon nanomaterial. This is because the distance between the detached antibody and carbon nanomaterial is farther than 10 nm, which is the maximum distance for the chemiluminescence (or fluorescence) resonance energy transfer between a bio- or chemical material and a carbon nanomaterial. Thus, the strength of signal is dependent on the concentration of antigen in a sample. In other words, the strength of signal is proportionally enhanced with the increase of antigen in a sample. Based on the phenomena shown in FIG. 1B, the inventor of the present disclosure invented the novel immunoassay as described in the present disclosure. For the purpose of distinguishing this novel immunoassay from the conventional immunoassay techniques, the novel immunoassay according to the present disclosure will be referred as “all-in-one immunoassay” in this specification.

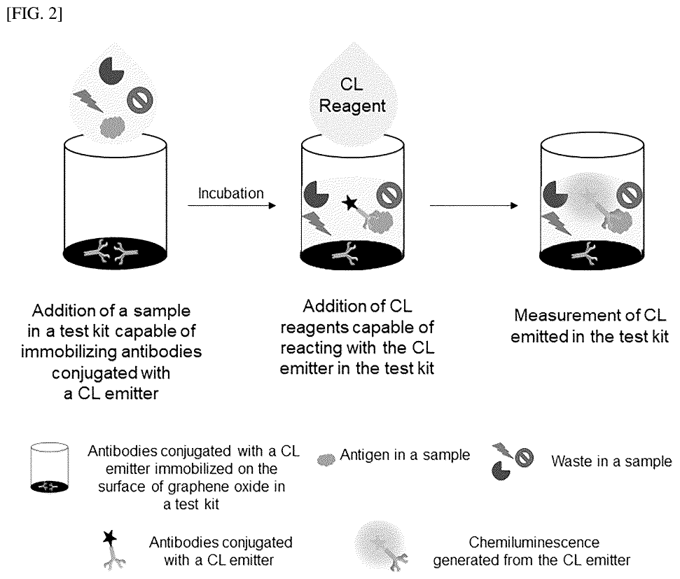

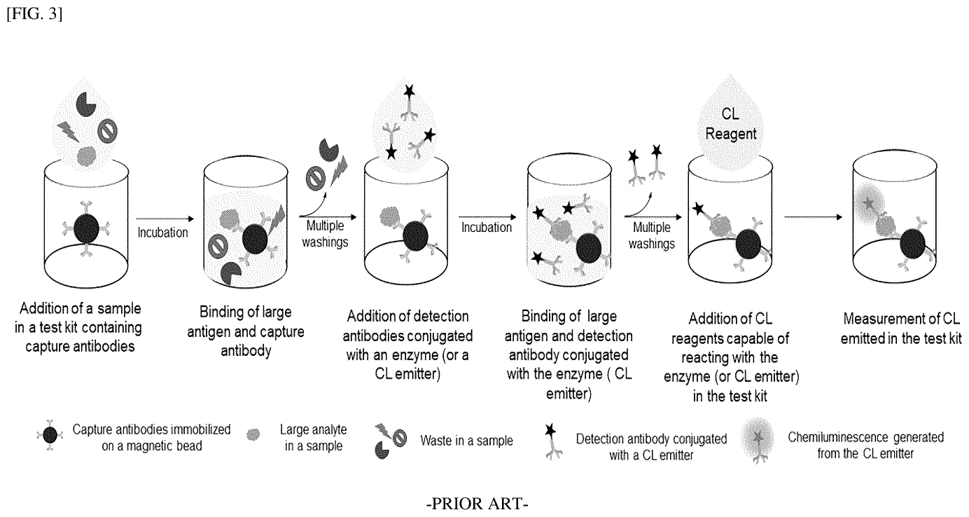

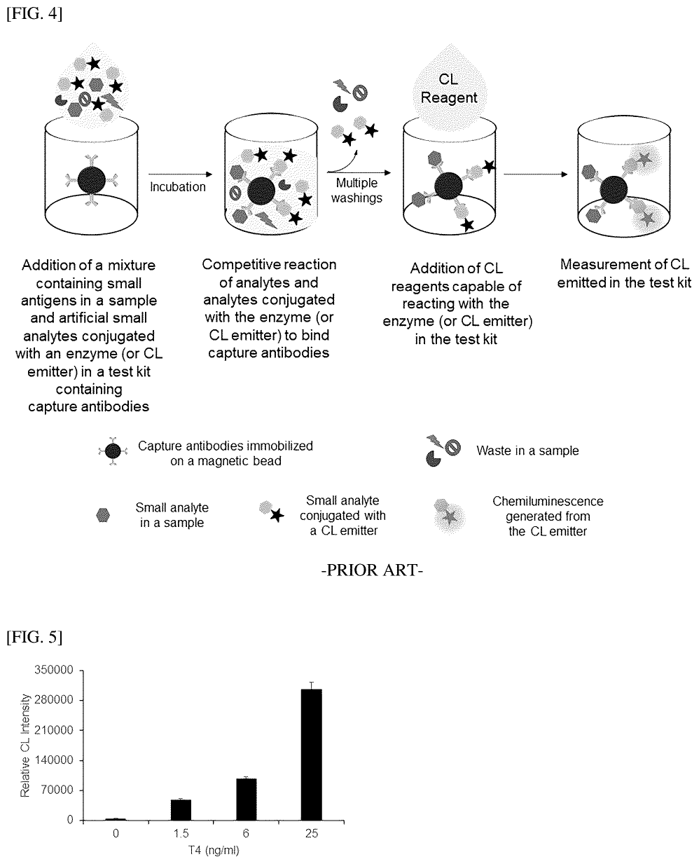

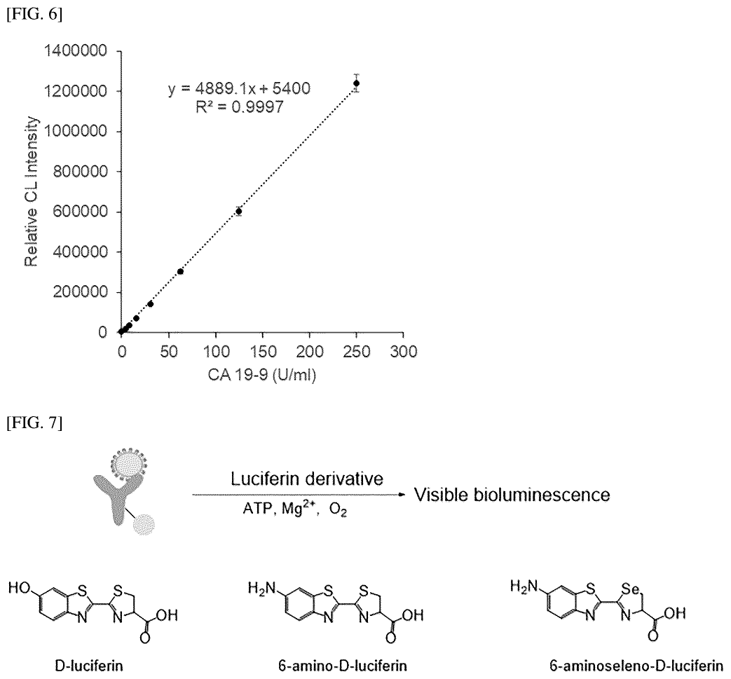

The all-in-one immunoassay shown in FIG. 2 can rapidly quantify an antigen in a sample without time-consuming procedures (e.g., multiple incubations and washings) required by conventional methods such as competitive and sandwich immunoassays shown in FIGS. 3 and 4. FIGS. 2-4 are examples that show the comparison between the all-in-one immunoassay with conventional immunoassays using a chemiluminescence detection such as alkaline phosphatase, horseradish peroxidase (HRP), or acridinium ester chemiluminescence detection. Also, the all-in-one immunoassay can be used with various detection methods, such as bioluminescence, chemiluminescence, colorimetric, electrochemical, electrochemiluminescence, fluorescence, and radiometry with the replacement of bio- or chemical material labeled antibody. In conclusion, the analytical time and sensitivity of the all-in-one immunoassay with a specific antibody conjugated with bio- or chemical materials, which are used to generate a signal such as bioluminescence, chemiluminescence, colorimetric, electrochemical, electrochemiluminescence, fluorescence, and radiometry, were faster and better than those of the conventional immunoassays. This is because the all-in-one immunoassay is operated without adding (1) artificially manufactured antigen conjugated with bio- or chemical materials for the competitive immunoassay and (2) a detection antibody conjugated with bio- or chemical materials (i.e., marker) for the sandwich immunoassay. Also, signal measured from the all-in-one immunoassay is always enhanced with the increase of a specific antigen concentration in a sample because the antibody concentration detached from the surface of carbon nanomaterial is proportional to the number of antigens in a sample. For example, 1,1′-oxalyldiimidazole (ODI) chemiluminescence emitted from the rapid all-in-one immunoassay using HRP labeled antibodies weakly immobilized on the surface of graphene oxide film was enhanced with the increase of thyroxine (T4) in a sample, as shown in FIG. 5, whereas that generated from the time-consuming competitive immunoassay with luminol chemiluminescence detection, applied as a conventional immunoassay for the analysis of small molecules such as T4, decreased exponentially with the increase of T4 concentration. The all-in-one immunoassay, capable of quantifying T4 within 25 min, was more sensitive than the commercially available competitive immunoassay (T4 AccuLite CLIA, Monobind, Inc) operated with a 50 min incubation and multiple washings. Also, the analytical time for the quantification of CA 19-9, which is a tumor marker, using the all-in-one immunoassay with acridinium ester chemiluminescence detection was as short as 30 min. On the other hand, a commercially available conventional sandwich immunoassay with luminol chemiluminescence detection (CA 19-9 AccuLite CLIA kit, Monobind, Inc) requires multiple incubations that take a total of 80 min and several time-consuming washings to analyze CA 19-9. The sensitivity of the all-in-one immunoassay with good linear calibration curve shown in FIG. 6 was better than that of the time-consuming sandwich immunoassay in quantifying CA 19-9 in human serum.

The all-in-one immunoassay can be operated with all detection methods applied for conventional competitive and sandwich immunoassays. Thus, antigens bound with antibodies labeled with bio- or chemical materials obtained from the all-in-one immunoassay can be quantified using various detection methods such as bioluminescence, chemiluminescence, colorimetric, electrochemical, electrochemiluminescence, naked-eye, fluorescence, and radiometry, as follows.

### A. Detection Methods Applied in the All-in-One Immunoassay

A specific antibody used for the all-in-one immunoassay using a detection method was conjugated with a marker, capable of generating a signal, based on one of several antibody labeling methods such as NHS (Succinimidyl) ester method, carbodiimide method, and periodate method. These methods to labeling a marker into an antibody are well known in the art, for example, in the following documents, which are incorporated herein by reference.
E. Ishikawa, in Immunoassay, eds. E. P. Diamandis and T. K. Christopoulos, Academic Press, San Diego, 1996, DOI: https://doi.org/10.1016/B978-012214730-2/50009-1. pp. 191-204.
H. Jiang, G. D. D'Agostino, P. A. Cole and D. R. Dempsey, in Methods in Enzymology, ed. D. M. Chenoweth, Academic Press, 2020, vol. 639, pp. 333-353.
   1. All-in-One Immunoassay with Bioluminescence Detection

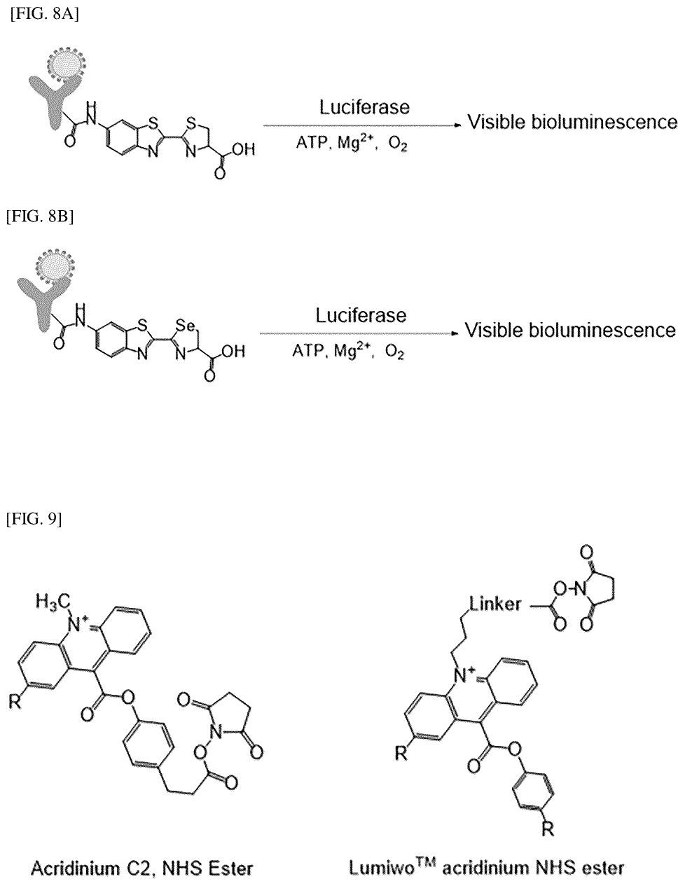

As shown in FIGS. 7, 8A and 8B, the antibody for the all-in-one immunoassay can be conjugated with luciferase or luciferin derivative such as 6-amino-D-luciferin, and 6-aminoseleno-D-luciferin can emit bioluminescence in the presence of an antigen capable of strongly binding with the antibody. The strength of bioluminescence emitted through the all-in-one immunoassay is enhanced with the increase of the antigen in a sample. For example, the antigen bound with the antibody conjugated with luciferase emit bright bioluminescence with the addition of a luciferin derivative, Mg²⁺, and adenosine triphosphate (ATP) in Tris-HCl (50 mM, pH 8.5). The color of bioluminescence in the all-in-one immunoassay was determined by the properties of luciferin derivative. The bioluminescence in the presence of D-luciferin potassium, 6-amino-D-luciferin, or 6-aminoseleno-D-luciferin was yellow, yellow-orange, or red. Also, the antigen bound with the antibody conjugated with 6-amino-D-luciferin or 6-aminoseleno-D-luciferin emitted yellow-orange or red bioluminescence with the addition of a certain concentrations of luciferase, Mg²⁺, and ATP in Tris-HCl (50 mM, pH 8.5). The concentrations of reagents added to generate bioluminescence in the all-in-one immunoassay were adjusted to quantify a target antigen with good accuracy, precision, and reliability. For example, 0.5 mM of 6-amino-D-luciferin, 10 mM of Mg²⁺, and 5 mM of ATP were used to quantify trace levels of D-dimer in human serum. Bioluminescence in the all-in-one immunoassay was immediately measured for 5 sec with the addition of the three reagents.

2. All-in-One Immunoassay with Chemiluminescence Detection
2.1 All-in-One Immunoassay with Acridinium Ester Chemiluminescence Detection

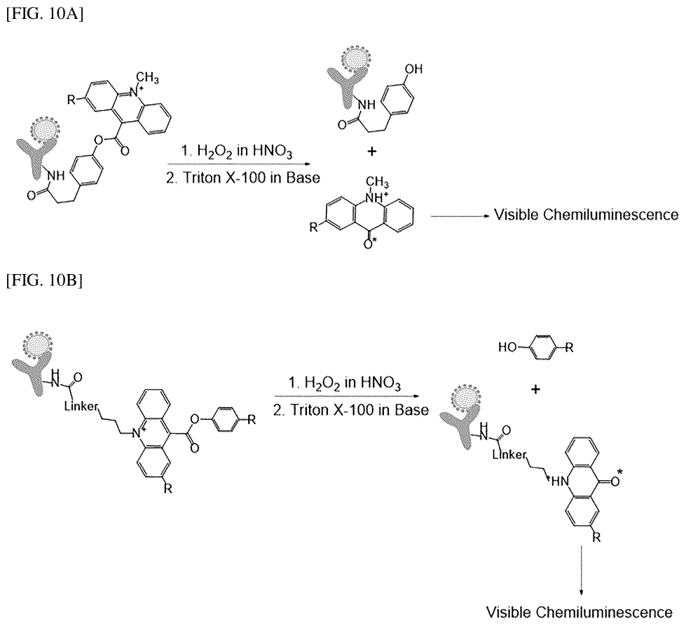

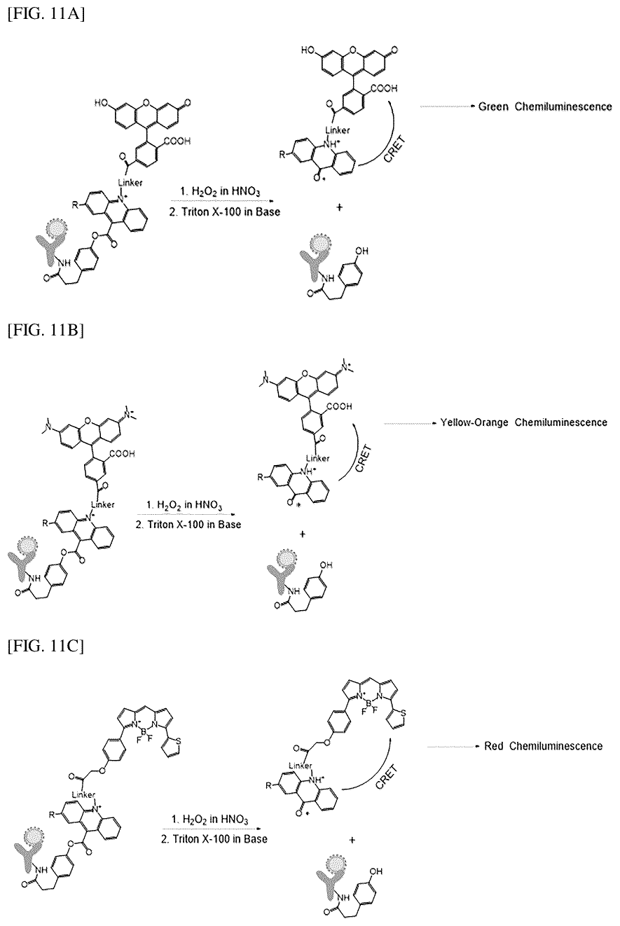

Several types of acridinium esters labeled antibody can be applied in the all-in-one immunoassay. For example, two different acridinium ester derivatives shown in FIG. 9 can be labeled in an antibody, as shown in FIGS. 10A and 10B. Both acridinium ester derivatives dissolved in DMSO can bind with primary amines of the antibody within 30 min at room temperature. As shown in FIG. 10A, the high-energy intermediate (or activator) formed with the consecutive additions of (1) H₂O₂ (5-40 mM) in HNO₃ (25-100 mM) and (2) NaOH (0.1-0.5 N) containing trace concentration of Triton X-100 is separated from the antibody. Then, the high-energy intermediate emits bright blue chemiluminescence. Also, the high-energy intermediate bound with the antibody after the consecutive additions of two reagents is formed as shown in FIG. 10B. The high-energy intermediate also emits blue chemiluminescence. For example, acridinium ester chemiluminescence in the all-in-one immunoassay operated based on the reaction mechanism shown in FIGS. 10A and 10B were proportionally enhanced with the increase of D-dimer in human serum. The emission wavelength of the all-in-one immunoassay using an acridinium ester links with a luminescent dye (e.g., fluorescein, rhodamine B, BDP) is longer than that using the acridinium C₂ NHS ester based on the chemiluminescence resonance energy transfer (CRET) from the high-energy intermediate formed with the chemical reaction to the luminescent dye. Thus, the color of chemiluminescence emitted from an acridinium ester linked with a luminescent dye in the all-in-one immunoassay is determined by the emission wavelength of the luminescent dye. In conclusion, the all-in-one immunoassay can simultaneously analyze multiple target antigens existing in a sample using antibodies bound with 2-4 luminescent dyes capable of emitting different colors of chemiluminescence such as blue, green, yellow-orange, and red. In other words, the all-in-one immunoassay using acridinium ester linked luminescent dyes can be applied as a new multiplex assay for the simultaneous quantifications of several target antigens in a sample.

2.2. All-in-One Immunoassay with Chemiluminescence Detection using Horseradish peroxidase
2.2.1. All-in-One Immunoassay with Luminol Chemiluminescence Detection

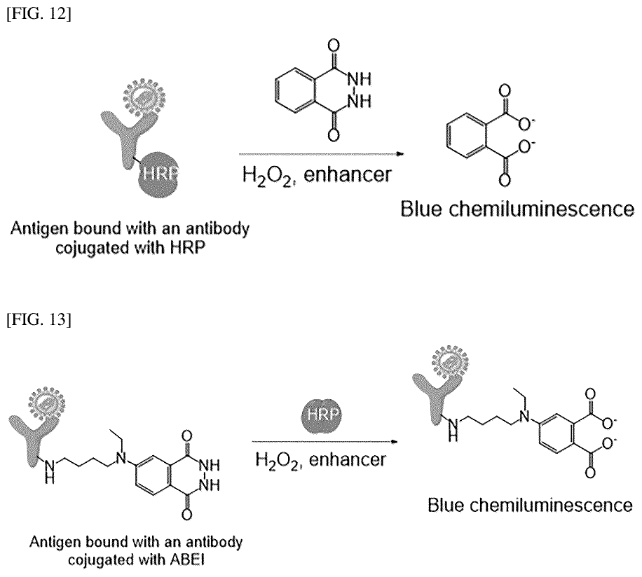

Luminol chemiluminescence can be applied to as a detection method of the all-in-one immunoassay. As shown in FIG. 12, an antibody conjugated with horseradish peroxidase (HRP) emits blue chemiluminescence with the addition of luminol (or isoluminol, 0.1-2 mM) and H₂O₂ (1-5 mM) in Tris-HCl (10-100 mM, pH 8-9) containing an enhancer, such as 4-iodophenol, 4-(17midazole-1-yl)phenol, 4-bromophenol, 4-phenylphenol, 3-(10′-phenothiazinyl) propane-1-sulfonate, and 4-morpholinopyridine). Also, the all-in-one immunoassay can be operated with an antibody conjugated with N-(4-aminobutyl)-N-ethylisoluminol (ABEI) to emit bright luminol chemiluminescence with the addition of HRP (0.5-10 U/ml), H₂O₂ (1-10 mM) in Tris-HCl (10-100 mM, pH 8-9) containing an enhancer (e.g., 4-iodophenol, 4-(imidazole-1-yl)phenol, 4-bromophenol, 4-phenylphenol, 3-(10′-phenothiazinyl) propane-1-sulfonate, 4-morpholinopyridine) as shown in FIG. 13. As examples, trace levels of D-dimer in human serum were quantified with the all-in-one immunoassay using the two different luminol chemiluminescence detections shown in FIGS. 12 and 13.

2.2.2. All-in-One Immunoassay with 1,1′-Oxalyldiimidazole Chemiluminescence Detection Operated with HRP Enzyme Reaction

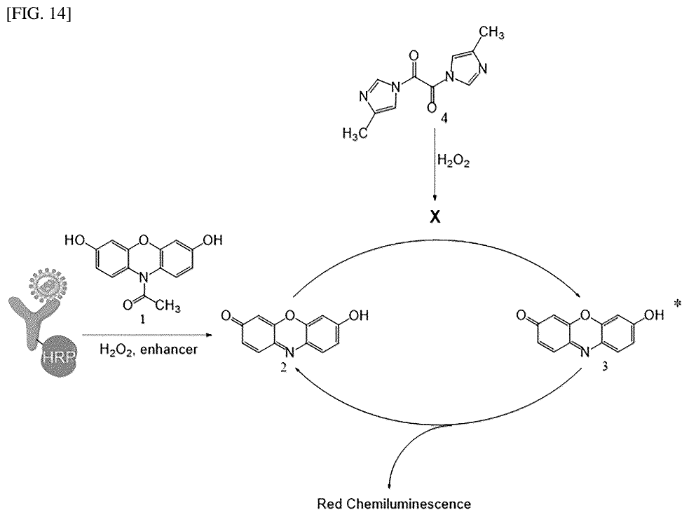

As shown in FIG. 14, an antigen bound with an antibody-conjugated HRP can be quantified with the all-in-one immunoassay with 1,1′-oxalyldiimidazole (ODI) chemiluminescence detection. The HRP labeled antibody reacts with Amplex Red (non-luminescent dye, 5-50 μM) in the presence of H₂O₂ (0.5-2 mM) and an enhancer (e.g., 4-iodophenol, 4-(imidazole-1-yl)phenol, 4-bromophenol, 4-phenylphenol) in Tris-HCl (5-100 mM, pH 7-8.5) or PBS (10 mM, pH 7.4) to produce resorufin which is a luminescent dye). The incubation time range (1-10 min) for the reaction is dependent on the experimental condition of the all-in-one immunoassay. For example, the incubation time for the quantification of the receptor-binding domain (RBD) of COVID-19 in a nasal specimen was 5 min, whereas that for the quantification of D-dimer in human serum was 3 min. Resorufin formed from the HRP enzyme reaction shown in FIG. 14 emits bright red chemiluminescence with the consecutive additions of H₂O₂ (20-100 mM) in isopropyl alcohol and ODI formed from the reaction of bis(2,4,6-trichlorophenyl) oxalate (TCPO, 10-100 μM) and an imidazole derivative (20-400 μM), such as imidazole, 2-methylimidazole, 2,4-dimethylimidazole, 4-methylimidazole, and 2-ethylimidazole in ethyl acetate. The brightness of chemiluminescence emitted in the all-in-one immunoassay with ODI-CL detection is enhanced with the increase of a target antigen concentration in a sample.

2.3. All-in-One Immunoassay with Chemiluminescence Detection using Alkaline Phosphatase (ALP) Labeled Antibody
2.3.1. All-in-One Immunoassay using Chemiluminescence Emitted from Substrate Triggered by Alkaline Phosphatase Labeled Antibody

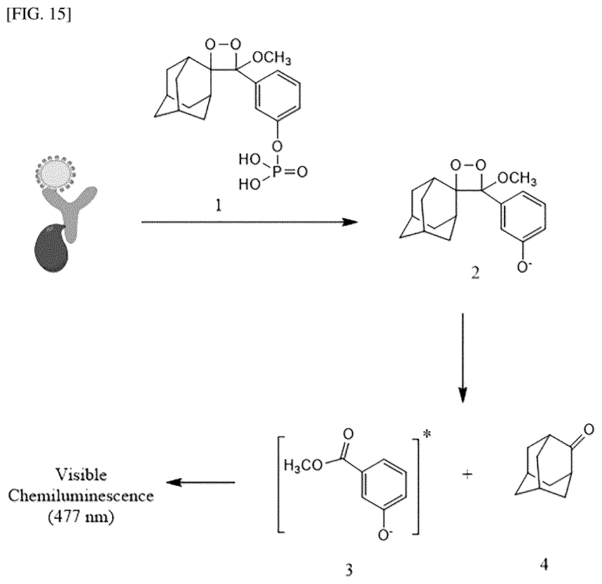

An adamantyl 1,2-dioxetane phosphate derivative, such as adamantyl 1,2-dioxetane aryl phosphate (AMPPD) and adamantyl 1,2-dioxetane chlorophenyl phosphate (CSPD), reacts with alkaline phosphatase (ALP) under basic condition (pH 9-11) to emit glow chemiluminescence. As shown in FIG. 15, AMPPD, 1, reacts with ALP-labeled antibody-bound antigen formed in the all-in-one immunoassay to produce the intermediate 2. Then, the intermediate, 2, is rapidly separated to the high-energy-intermediate, 3, under the excited state and a stable product, 4. Finally, the high-energy intermediate, 3, emits blue chemiluminescence. For example, trace levels of D-dimer in human serum were quantified with the all-in-one immunoassay with chemiluminescence detection operated based on the mechanism shown in FIG. 15. The brightness of chemiluminescence emitted through the all-in-one immunoassay was enhanced with the increase of D-dimer in human serum. The mixture of antigen bound with antibody-conjugated ALP and a CSPD derivative was incubated for 5 min. Then, the strength of chemiluminescence emitted in a detection cell was measured for 5 sec with good reliability.

2.3.2. All-in-One Immunoassay with ODI Chemiluminescence Detection Operated with ALP Enzyme Reaction

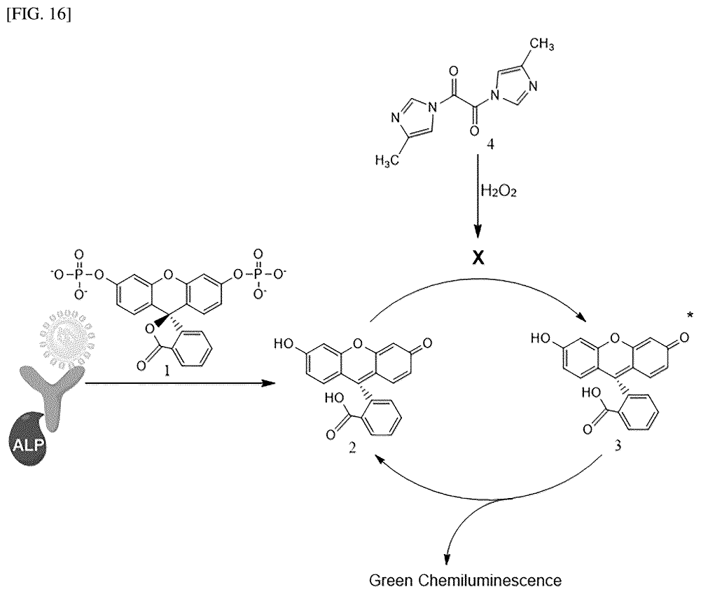

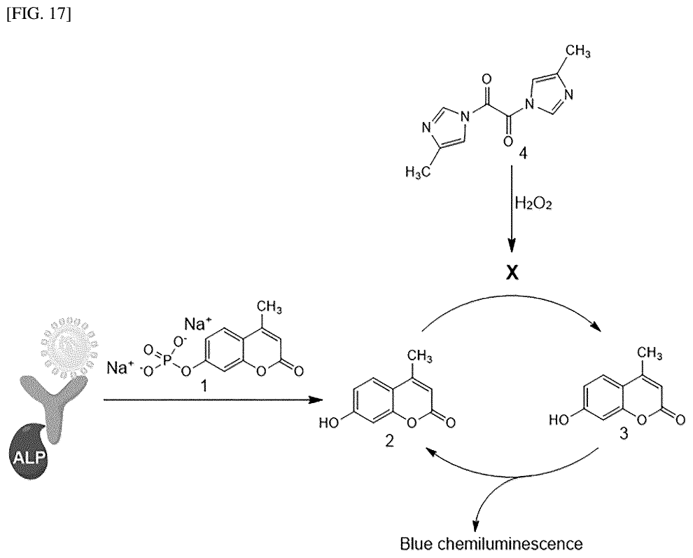

Fluorescein diphosphate (FDP) and 4-methylunbelliferyl phosphate (4-MUP) are non-luminescent compounds. However, FDP and 4-MUP in the presence of alkaline phosphatase (ALP) is hydrolyzed to produce fluorescein and 4-methylunbelliferon (4-MU), which are strong luminescent dyes. Yields of fluorescein and 4-MU transformed from FDP and 4-MUP are dependent on the activity of ALP added for the enzyme reaction. Thus, the all-in-one immunoassay with ODI chemiluminescence detection can be applied to quantify a target antigen in a sample based on the mechanisms shown in FIGS. 16 and 17. The color of ODI chemiluminescence is dependent on the property of luminescent dye from the ALP enzyme reaction in the presence of a non-luminescent compound. For example, trace levels of D-dimer in human serum were quantified with an antibody-conjugated ALP, capable of specifically capturing D-dimer in a sample, FDP, and ODI chemiluminescence detection. After the binding reaction between D-dimer and the antibody conjugated with ALP for 15 min in an all-in-one immunoassay kit, FDP (0.1-0.5 mM) in Tris-HCl (10 mM, pH 8.5) was placed in the kit to produce fluorescein for 5 min. After the final incubation, ODI chemiluminescence emitted from each sample was measured for 2 sec with the consecutive additions of H₂O₂ (20-100 mM) in isopropyl alcohol and ODI, which was formed from the reaction of bis(2,4,6-trichlorophenyl) oxalate (TCPO, 10-100 μM) and an imidazole derivative (20-400 μM) such as imidazole, 2-methylimidazole, 2,4-dimethylimidazole, 4-methylimidazole, and 2-ethylimidazole in ethyl acetate. The relative chemiluminescence intensity was proportionally enhanced with the increase of D-dimer concentration in human serum.

2.4. All-in-one immunoassay with phenylglyoxal derivative chemiluminescence detection

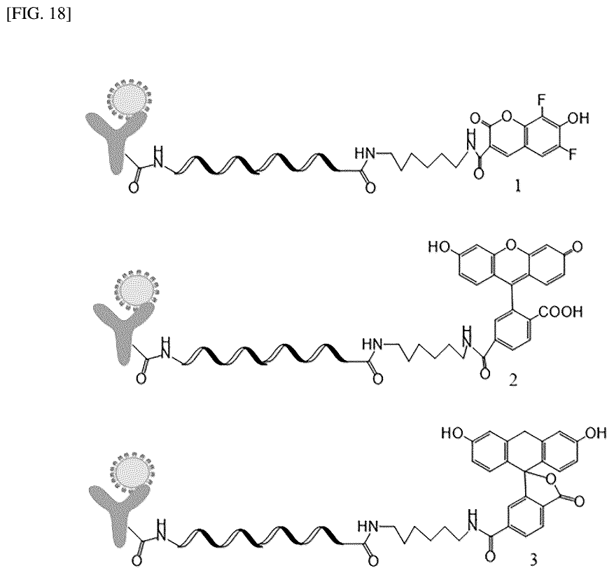

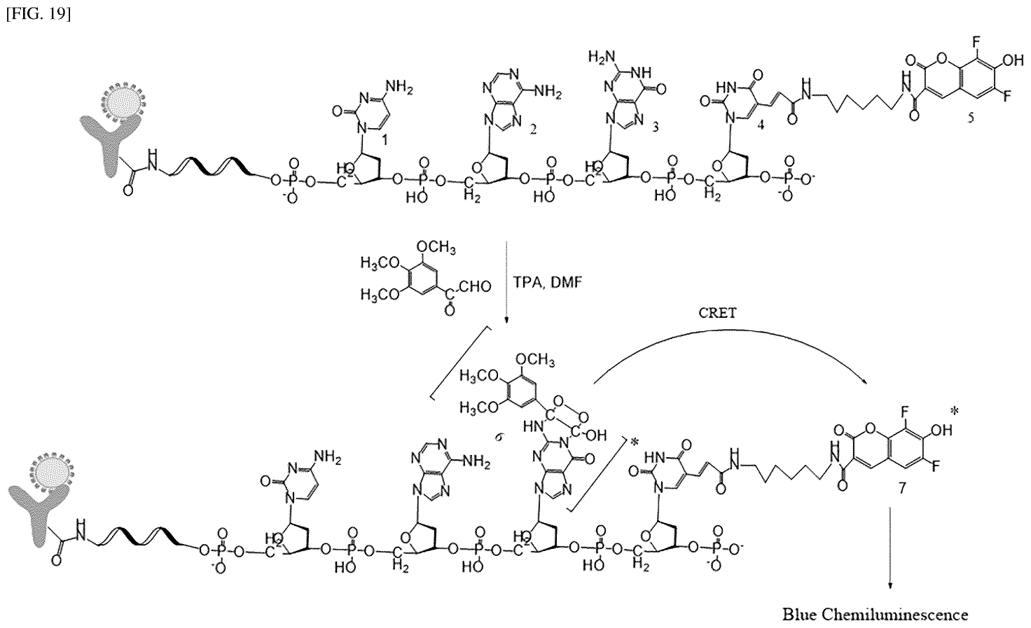

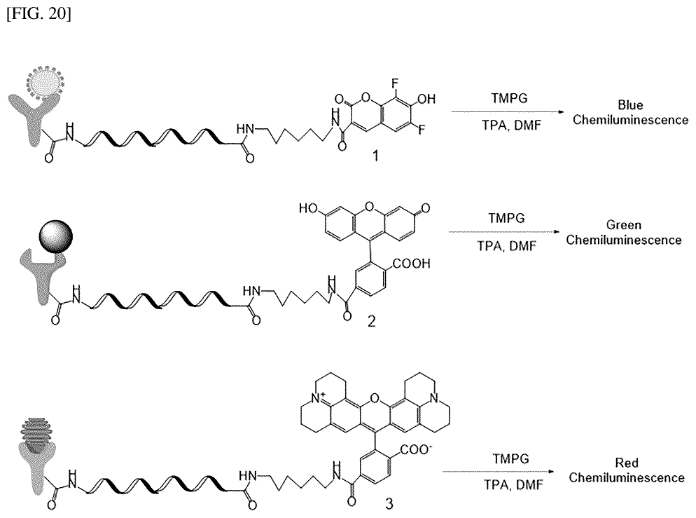

A phenylglyoxal derivative, such as 3-methoxylphenylglyoxal (3-MPG) and 3, 4, 5-trimethoxylphenylglyoxal (TMPG), reacts with guanine of single stranded DNA (or RNA) to produce a high-energy intermediate capable of emitting dim chemiluminescence by itself, as well as transferring energy to a luminescent dye based on the principle of chemiluminescence resonance energy transfer (CRET). The luminescent dye excited due to the CRET emits bright chemiluminescence. The brightness and color of chemiluminescence emitted in this reaction is dependent on the properties of luminescent dye excited due to the CRET. Based on the principle, the all-in-one immunoassay with phenylglyoxal derivative chemiluminescence detection uses an antibody conjugated with a single stranded DNA (or RNA) bound with a luminescent dye, as shown in FIG. 18. The single stranded DNA (or RNA) must have multiple guanines (5-15) with other components (e.g., adenine, cytosine, and thymine for DNA, adenine, cytosine, uracil for RNA) to produce a strong high-energy intermediate with the reaction of guanine and a phenylglyoxal derivative. Also, guanines of the single stranded DNA (or RNA) should be adjacent to the luminescent dye labeled with a single stranded DNA (or RNA) to transfer stronger energy to the luminescent dye from the high-energy intermediates formed from the reaction of multiple guanines and a phenylglyoxal derivative. As a result, the all-in-one immunoassay with chemiluminescence detection with a single stranded DNA (20-100 bp) conjugated with a luminescent dye and a phenylglyoxal derivative was invented. For example, trace levels of D-dimer in human serum were quantified with the all-in-one immunoassay with chemiluminescence detection operated with a single-stranded DNA (30 bp) conjugated with Pacific Blue, TMPG (1-10 mM) in N, N-dimethylformamide (DMF), and tetra-n-propylammonium hydroxide (TPA, 15-30 mM in deionized water) based on the reaction mechanism shown in FIG. 19. In addition, the all-in-one immunoassay with a phenylglyoxal derivative chemiluminescence detection can be used as a multiplex immunoassay tool. For example, three different target antigens existing in a sample can be simultaneously quantified with the multiplex all-in-one immunoassay capable of detecting three different color lights using a luminometer with three photon-multiplier tubes to filter and measure a specific color of chemiluminescence as shown in FIG. 20. Each antibody having a good specificity to capture a target antigen in a sample was conjugated with different luminescence dye to emit a distinctive color of chemiluminescence.

2.5 All-in-One Immunoassay with ODI Chemiluminescence Detection Operated with an Antibody Conjugated with Luminescent Dyes

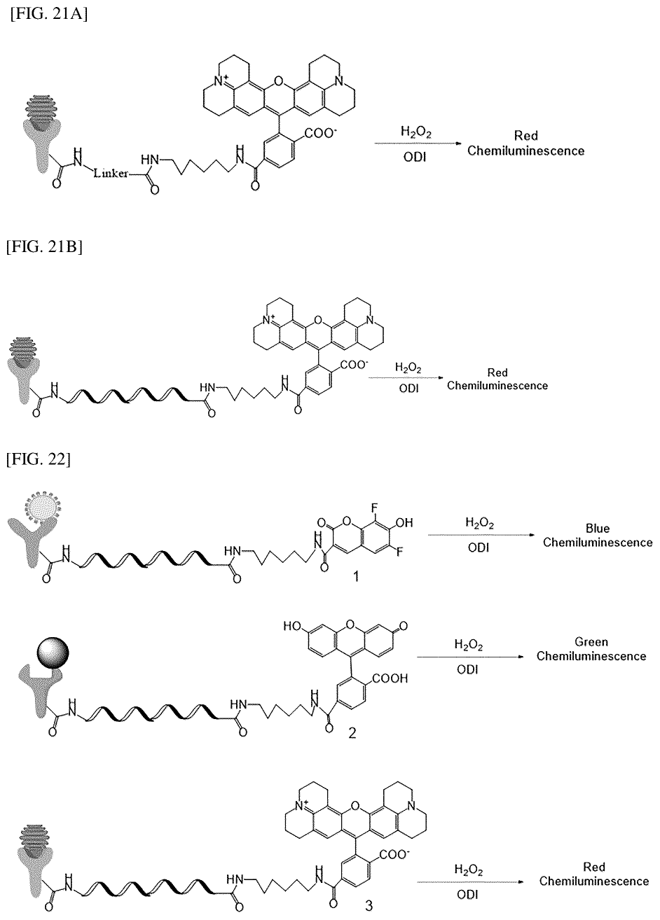

As shown in FIGS. 14, 16, and 17, a luminescent dye emits a distinctive color of chemiluminescence with the addition of H₂O₂ and an ODI derivative. Thus, the all-in-one immunoassay with ODI chemiluminescence detection was operated with an antibody conjugated with luminescent dyes. For example, the all-in-one immunoassay quantified trace levels of D-dimer in human serum based on the reaction mechanism shown in FIGS. 21A and 21B. A human serum sample containing D-dimer was incubated with antibodies conjugated with rhodamine 101 through a linker or single stranded DNA (30-100 bp) for 15 min at room temperature. Then, the D-dimer-bound antibody-conjugated rhodamine 101 emitted red chemiluminescence with the addition of H₂O₂ in isopropyl alcohol and ODI in ethyl acetate. The distance between the antibody and rhodamine 101 connected through the single stranded DNA was farther than that conjugated through the linker. Thus, the all-in-one immunoassay using the D-dimer antibodies conjugated with rhodamine 101 through the single stranded DNA was more sensitive than that through the linker because rhodamine 101 under the former condition can emit brighter chemiluminescence without the interference effect by the large size D-dimer antibody. Also, the all-in-one immunoassay with ODI chemiluminescence detection operated using antibodies conjugated with luminescent dyes can be used as a multiplex all-in-one immunoassay capable of simultaneously quantifying different target antigens existing in a sample shown in FIG. 22. FIGS. 21A, 21B and 22 indicate that the all-in-one immunoassay with ODI chemiluminescence detection can be operated with an antibody conjugated with alternative luminescent materials such as luminescent nanospheres and luminescent carbon dots instead of small luminescent dyes.

3. All-in-One Immunoassay with Colorimetric Detection
3.1 All-on-One Immunoassay with Colorimetric Detection Operated with Antigen-Bound Antibody Conjugated with HRP

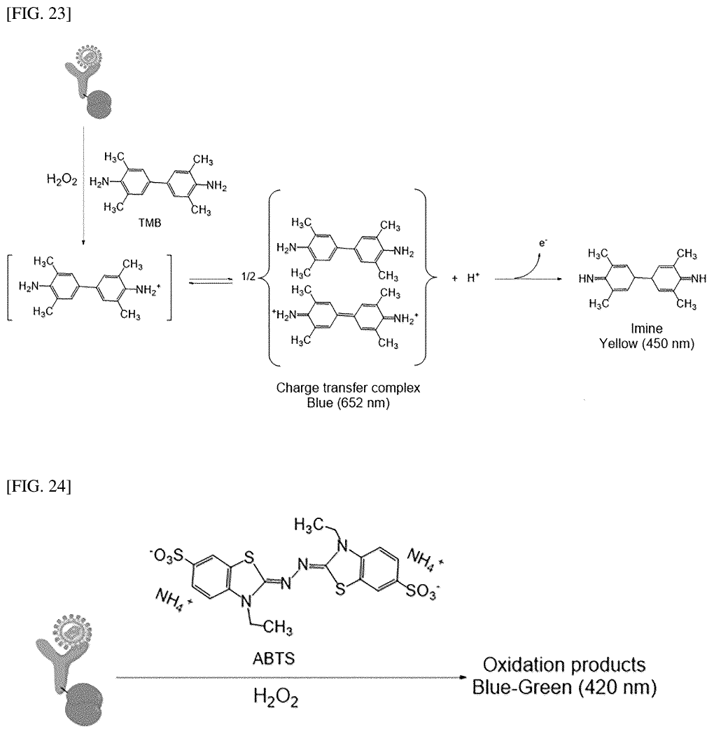

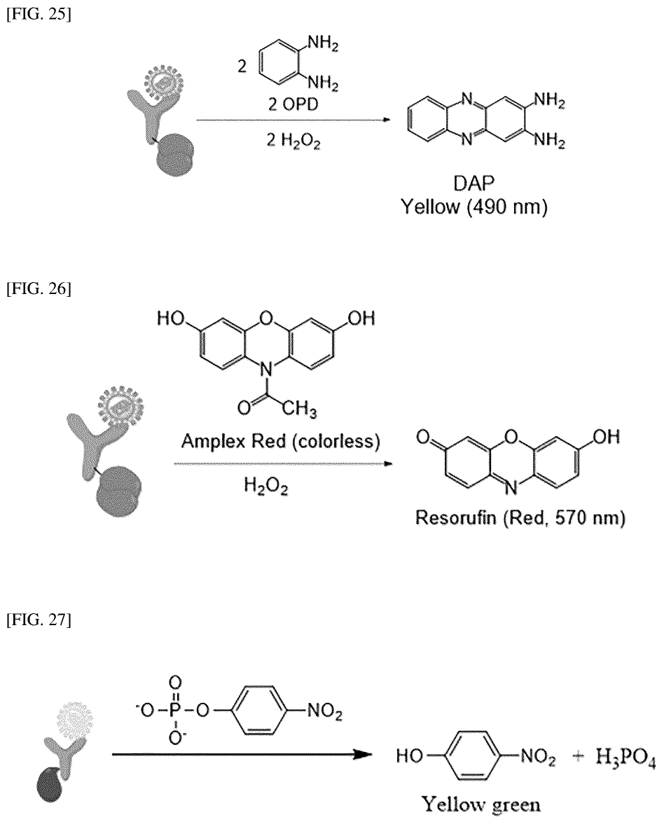

As shown in FIGS. 23-26, several different types of substrates can be used for the all-in-one immunoassay with a colorimetric detection using HRP labeled antibody capable of specifically capturing a target antigen in a sample. Thus, the sensitivity of the all-in-one immunoassay with a colorimetric detection is dependent on the properties of a substrate added for the enzyme reaction in the presence of HRP and H₂O₂. Also, the sensitivity of the all-in-one immunoassay is dependent on the incubation time for the HRP enzyme reaction. However, the incubation time for the all-in-one immunoassay is much shorter than those for the conventional immunoassays (e.g., competitive- and sandwich-immunoassay). For example, D-dimer in human serum was quantified using the all-in-one immunoassay with a colorimetric detection within 30 min, without no washing. However, quantifying it with a commercially available sandwich enzyme-linked immunosorbent assay (ELISA, abcam) using a HRP labeled detection antibody required a 90-min incubation and time-consuming washings. Also, the detection limit (1.2 ng/ml) of the rapid all-in-one immunoassay was slightly lower than that of the complicated and time-consuming ELISA.

3.2 All-on-One Immunoassay with Colorimetric Detection Operated with Antigen-Bound Antibody Conjugated with ALP

Using pNPP (p-Nitrophenyl Phosphate, Disodium Salt) as a substrate of ALP labeled antibody, the all-in-one immunoassay with a colorimetric detection can be applied for the rapid quantification of a target antigen in a sample as shown in FIG. 27. For example, trace levels of D-dimer in human serum were quantified with the all-in-one immunoassay within 30 min. The detection limit (3.5 ng/ml) of the all-in-one immunoassay with ALP-labeled antibody using pNPP was slightly higher than that (1.2 ng/ml) of the all-in-one immunoassay with HRP-labeled antibody using TMB. However, the all-in-one immunoassay with ALP-labeled antibody using pNPP can also be applied for the quantification of D-dimer because the normal range of D-dimer in human serum is as high as 250 ng/ml.

3.3 All-on-One Immunoassay with Colorimetric Detection Operated with Antigen-Bound Antibody Conjugated with Other Enzymes Instead of Conventional Enzymes Such as ALP and HRP

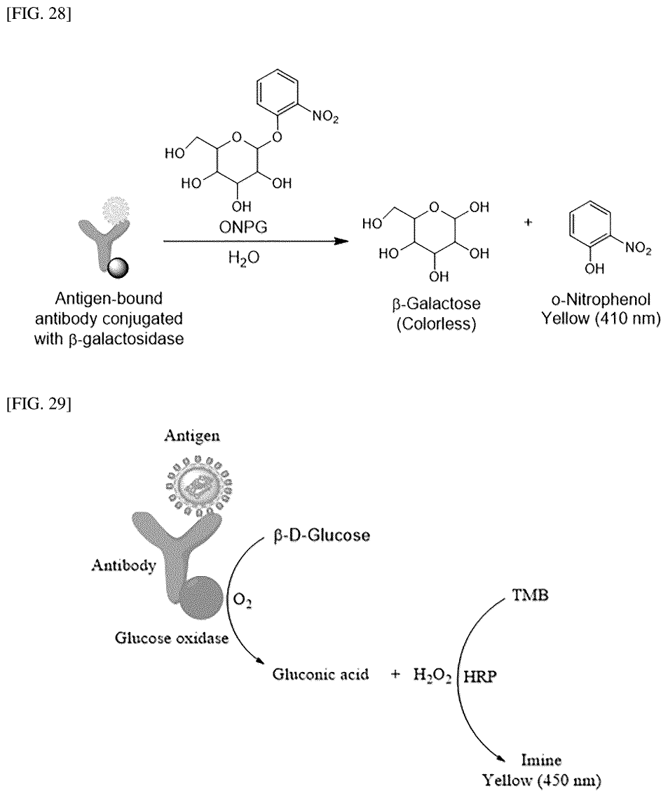

The all-in-one immunoassay with a colorimetric detection can be operated with an antibody conjugated with other enzymes instead of HRP and ALP as shown in FIGS. 28 and 29. The sensitivity of the all-in-one immunoassay with a colorimetric detection is determined by the property of an alternative enzyme (e.g., β-galactosidase, glucose oxidase) instead of the conventional enzymes. Also, the incubation time for the enzyme reaction is dependent on the properties of the alternative enzyme. Substrates used for the all-in-one immunoassay with a colorimetric detection using a glucose oxidase labeled antibody are the same as those (e.g., TMB, ABTS, OPD, Amplex Red) using HRP labeled antibody.

4. All-on-one immunoassay with an electrochemical detection operated with antigen-bound antibody conjugated with an enzyme

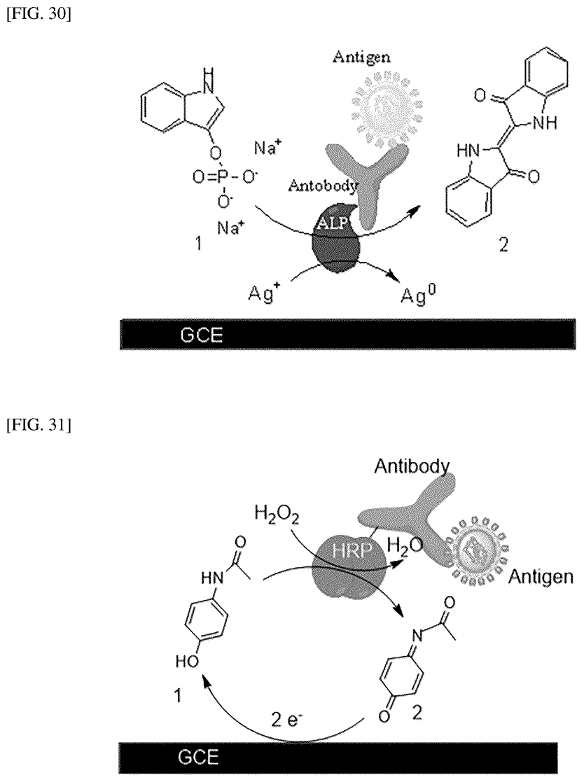

As shown in FIGS. 30 and 31, the all-in-one immunoassay with an electrochemical detection, capable of measuring current, is operated with a conventional enzyme (e.g., ALP, HRP) labeled antibody bound with a target antigen. First, the target antigen interacts with the antibody-conjugated ALP (or HRP) weakly immobilized on the surface of graphene oxide film fixed on a well as shown in FIG. 2. After incubating for certain amount of time (10-30 min), a volume (e.g., of solution containing the antigen-bound antibody-conjugated ALP for HRP) was transferred from the well to an electrochemical cell having a glass carbon electrode. Then, an appropriate substrate capable of reacting with a specific enzyme, such as ALP, HRP, was added in the electrochemical cell. The final mixture was incubated for a certain amount of time (5-20 min). After the final incubation, the current generated from the electrochemical cell was measured. The difference between currents measured in the absence and presence of the target antigen was increased with the increase of the target antigen concentration. For example, trace levels of D-dimer in human serum were quantified with good reliability using the all-in-one immunoassay with an electrochemical detection that uses a specific antibody conjugated with HRP, H₂O₂, and acetaminophen as a substrate based on the mechanism shown in FIG. 31. The sensitivity and incubation time of the all-in-one immunoassay with an electrochemical detection are dependent on the types of substrate or electrode that are used.

5.0 All-in-One Immunoassay with an Electrochemiluminescence Detection

5.1. All-in-One Immunoassay with an Electrochemiluminescence Detection Operated with a Ruthenium Complex Labeled Antibody-Bound a Target Antigen

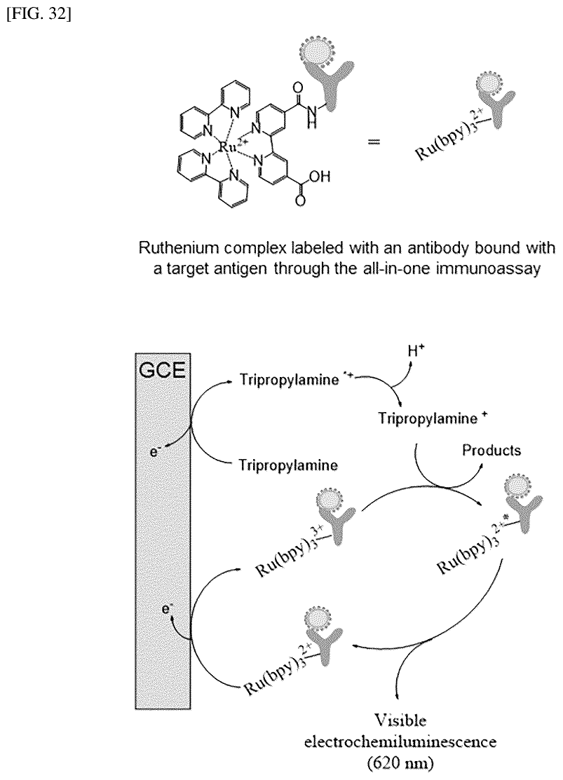

A ruthenium complex-labeled antibody-bound target antigen (Ru(bpy)₃²⁺<Antibody>Antigen) shown in the top of FIG. 32 is formed through the all-in-one immunoassay procedure (see FIG. 2). Small volume of solution containing Ru(bpy)₃²⁺<Antibody>Antigen formed from the first procedure of the all-in-one immunoassay shown in FIG. 2 is transferred to an electrochemiluminescence cell. The Ru(bpy)₃²⁺ is oxidized on the glass carbon electrode (GCE) to produce Ru(bpy)₃³⁺ as shown in the bottom of FIG. 32. Also, tripropylamine, which is a co-reagent for electrochemiluminescence, is also oxidized to form tripropylamine+on the GCE electrode. Then, the Ru(bpy)₃³⁺ reacts with the tripropylamine⁺ to produce a high-energy intermediate (Ru(bpy)₃²⁺*) existing in the excited state. Finally, strong electrochemiluminescence generated at 620 nm is measured using a luminometer with a photomultiplier tube (PM-tube) through the rapid transition from Ru(bpy)₃²⁺* in the excited state to Ru(bpy)₃²⁺ in the ground state. Thus, the brightness of electrochemiluminescence, in the all-in-one immunoassay using Ru(bpy)₃²⁺ as a labeling material, is proportionally enhanced with the increase of target antigen (e.g., virus, cell, protein, peptide, small molecule) in a sample. Meanwhile, the brightness of electrochemiluminescence in the conventional immunoassays is enhanced or decreased with the increase of target antigen because the sandwich immunoassay can quantify a large molecule of target antigen (e.g., virus, cell, protein, peptide) and the competitive immunoassay can analyze a small molecule of target antigen such as triiodothyronine (T3) and thyroxine (T4). For example, trace levels of D-dimer in human serum were quantified with the Ru(bpy)₃²⁺ complex electrochemiluminescence detection. With the increase of D-dimer concentrations, the brightness of electrochemiluminescence was proportionally enhanced.

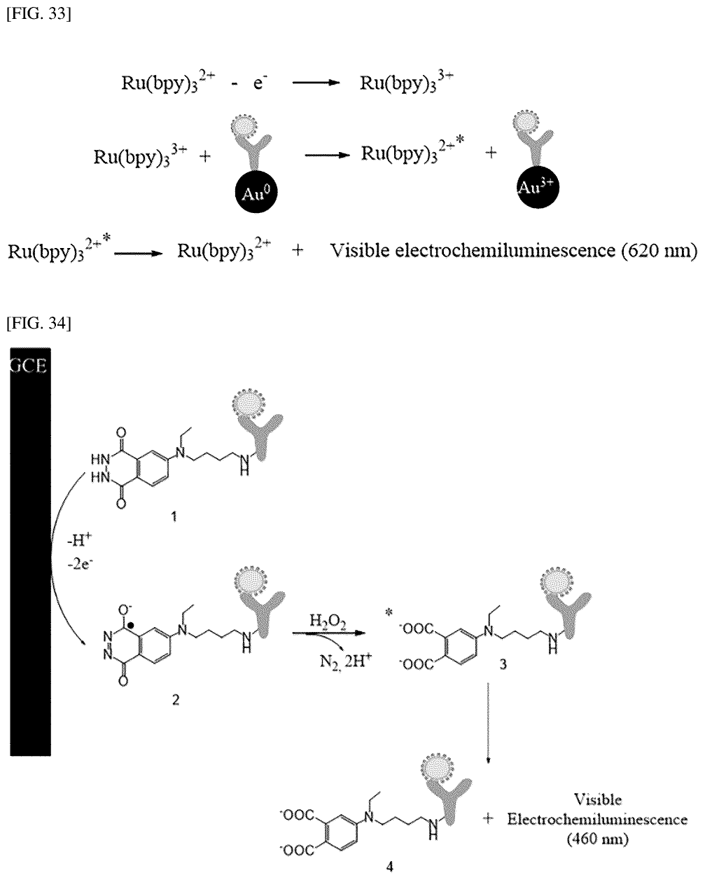

A nanoparticle (e.g., gold (Au), silver (Ag)) labeled antibody-bound target antigen formed from the all-in-one immunoassay can be applied to generate electrochemiluminescence emitted from Ru(bpy)₃²⁺* because the nanoparticle can act as a co-reagent, like tripropylamine, in the electrochemiluminescence reaction. For example, a gold nanoparticle labeled antibody-bound target antigen (Au<Antibody>Antigen) is formed from the first procedure of the all-in-one immunoassay shown in FIG. 2. A small volume of solution containing Au<Antibody>Antigen is transferred to an electrochemiluminescence cell. As shown in FIG. 33, Au labeled antibody-bound antigen can act as a co-reagent capable of reacting with Ru(bpy)₃³⁺ to produce Ru(bpy)₃²⁺*. The role of Au is the same as that of propylamine in the ruthenium complex electrochemiluminescence reaction. Finally, strong electrochemiluminescence is measured during the rapid transition of Ru(bpy)₃²⁺* in the excited state to Ru(bpy)₃²⁺ in the ground state shown in FIG. 33. Thus, the emission wavelength of the electrochemiluminescence emitted through the procedure shown in FIG. 33 is consistent with that generated through the mechanism shown in FIG. 32.

All-in-one immunoassay with an electrochemiluminescence detection operated with a ABEI labeled antibody-bound a target antigen

FIG. 34 shows that an ABEI-labeled antibody-bound target antigen (ABEI<Antibody>Antigen) formed from the first procedure for the all-in-one immunoassay shown in FIG. 2 can also generate strong electrochemiluminescence in an electrochemiluminescence cell. First, a small volume of ABEI<Antibody>Antigen is transferred to the electrochemiluminescence cell. The ABEI, 1, interacts with the GCE to produce the intermediate, 2. Then, 2 reacts with H₂O₂ to produce the high-energy intermediate, 3, in the excited state. Finally, strong electrochemiluminescence generated from the rapid transition from the excited state to the ground state is measured using a luminometer with a PM-tube. The brightness of the electrochemiluminescence is proportionally enhanced with increasing any size of target antigen (e.g., virus, cell, protein, peptide, small molecule) existing in a sample. For example, trace levels of D-dimer in human serum were quantified with the ABEI chemiluminescence detection. With the increase of D-dimer concentration, the brightness of electrochemiluminescence was proportionally enhanced.

6. All-in-One Immunoassay with Fluorescence Detection
6.1. All-in-One Immunoassay with Fluorescence Detection Using an Enzyme

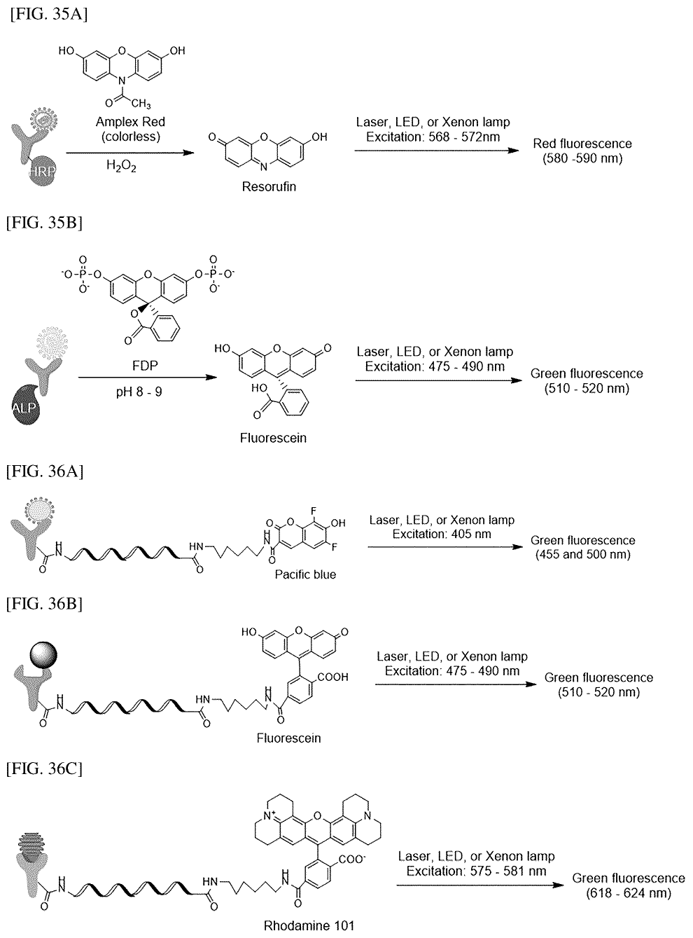

The all-in-one immunoassay can be operated with a fluorescence detection capable of sensing visible light emitted from fluorescence dye, formed from the reaction of a non-fluorescent substrate (e.g., FDP, Amplex Red) and an enzyme (e.g., ALP, HRP), excited by a light source such as laser, LED, or Xenon lamp. As shown in FIG. 35A, an enzyme labeled antibody-bound antigen (enzyme<antibody>antigen), formed from the first procedure of the all-in-one immunoassay (see FIG. 2), reacts with a substrate for a certain incubation time to produce a fluorescence dye. Then, the fluorescence dye excited by a light source emit a visible fluorescence for a certain incubation time. The color of fluorescence is dependent on the properties of the fluorescence dye. For example, trace levels of D-dimer in human serum were quantified with the all-in-one immunoassay with a fluorescence detection operated with HRP, Amplex Red, and H₂O₂. The detection limit of the all-in-one immunoassay with the fluorescence detection capable of quantifying D-dimer within 20 min was as low as 0.5 ng/ml, which is 500-fold lower than that of normal D-dimer concentration in human serum.

6.2. All-in-One Immunoassay with a Fluorescence Detection Using a Fluorescence Dye-Labeled Antibody.

The all-in-one immunoassay with a fluorescence detection can be also applied with a fluorescent dye-labeled antibody-bound antigen (fluorescent dye<antibody>antigen). As shown in FIG. 35B, a fluorescence<antibody>antigen, formed from the first procedure of the all-in-one immunoassay (see FIG. 2), emits visible fluorescence. The color of fluorescence emitted in the all-in-one immunoassay is dependent on the properties of a fluorescence dye as shown in FIGS. 36A, 36B and 36C. For example, D-dimer in human serum was quantified within 20 min with the all-in-one immunoassay with a fluorescence detection operated using fluorescein labeled antibody. The detection limit (8.2 ng/ml) of the all-in-one immunoassay using fluorescein-labeled antibodies was about 16-fold higher than that (0.5 ng/ml) of the all-in-one immunoassay using HRP-labeled antibodies even though the same fluorescence detection was used. This is because brightness of fluorescence emitted form a small fluorescence dye labeled large antibody is quenched due to the interference of the antibody. However, FIGS. 36A to 36C indicate that the all-in-one immunoassay with a fluorescence detection operated with a fluorescence<antibody>antigen can be applied as a multiplex assay method capable of simultaneously sensing different types of target antigens in a sample.

7. All-in-One Immunoassay Capable of Sensing a Positive or Negative Sample with the Naked Eye

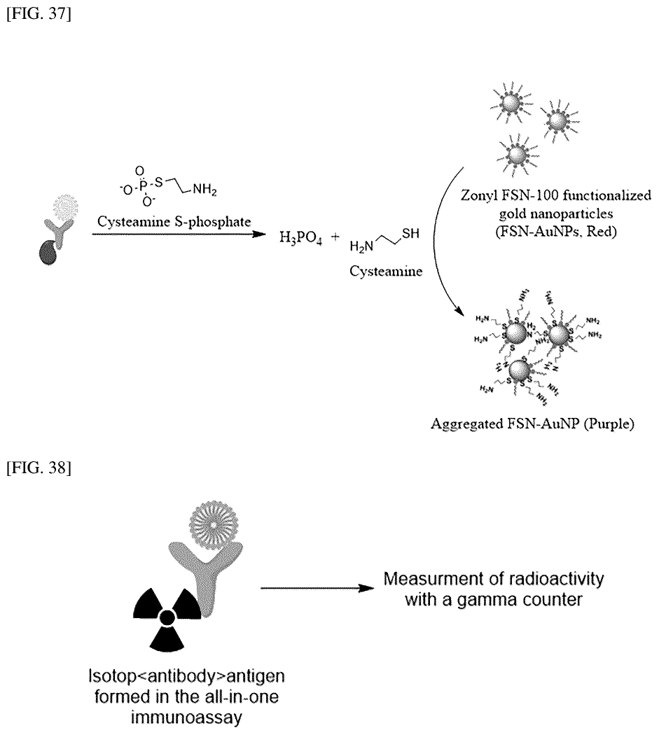

The all-in-one immunoassay can be applied to observe positive or negative results with the naked eye. For example, the color of Zonyl FSN-100 functionalized gold nanoparticles (FSN-AuNPs) changes from red to purple in the presence of cysteamine formed from the reaction of ALP-labeled antibody-bound antigen that is formed from the first procedure of the all-in-one immunoassay (see FIG. 2) and cysteamine S-phosphate, which is a substrate capable of reacting with ALP. The strength of purple shown with the aggregation of FSN-AuNPs is enhanced with the increase of the antigen concentration in a sample because cysteamine supports the aggregation of FSN-AuNPs. Thus, it is possible to develop an all-in-one immunoassay capable of sensing positive and negative samples with the naked eye based on the mechanism shown in FIG. 37.

8. All-in-One Immunoassay with a Radiometer

Among conventional radioimmunoassay methods, a competitive radioimmunoassay is operated with an antigen conjugated with an isotope (e.g., ¹²⁵I) whereas a sandwich radioimmunoassay is operated with a detection antibody conjugated with an isotope. For both the methods, time-consuming multi-washings and incubations are required like the complicated procedures of other conventional immunoassays shown in FIGS. 3 and 4. Also, the radioactivity measured with the competitive radioimmunoassay is exponentially decayed with the increase of a target antigens in a sample, whereas the radioactivity measured with the sandwich radioimmunoassay is proportionally enhanced with the increase of a target antigen concentration.

FIG. 38 shows that the all-in-one immunoassay with a radiometer can analyze target antigen in a sample with no washing and one-time incubation to produce an isotope-labeled antibody-bound antigen (isotope<antibody>antigen) formed in the presence of a target antigen in a sample. After the first procedure of the all-in-one immunoassay, the solution in the test kit needs to be transferred to a detection well to remove the relatively high background generated from the isotope-labeled antigen immobilized on the surface of the test kit after the incubation. The radioactivity of the solution in the absence and presence of isotope<antibody>antigen is measured with a gamma-counter. Thus, the radioactivity of solution in a detection well is proportionally enhanced with the increase of antigen concentration in a sample.

Hereinafter, the preparation of a device for the all-in-one immunoassay according to an exemplary embodiment of the present disclosure are described in detail through examples. However, the following examples are provided only to illustrate the present disclosure and the scope of the present disclosure is not limited thereto.

### B. Temporary Immobilization of Antibodies on the Surface of Carbon Nanomaterials

In order to operate the all-in-one immunoassay shown in FIG. 2, it is important to prepare antibodies temporarily attached to a surface of carbon nanomaterial based on the π-π stacking interaction between the antibody and the carbon material. Also, the removal of antibodies remaining in solution after the π-π stacking interaction is very important factor in reducing background measured in the absence of a target antigen as well as enhancing the sensitivity in the all-in-one immunoassay. There are two specific methods for preparing antibodies temporarily immobilized to a surface of carbon nanomaterial that can be applied as shown below.

### EXAMPLE 1

### All-in-One Immunoassay Operated with Antibodies Attached on the Surface of a Magnetic carbon Nanomaterial

### 1.1. Preparation of Magnetic Carbon Nanomaterials.

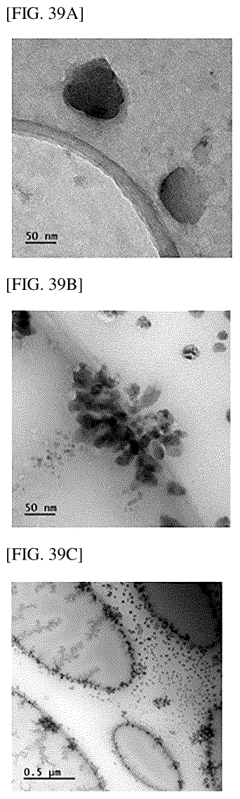

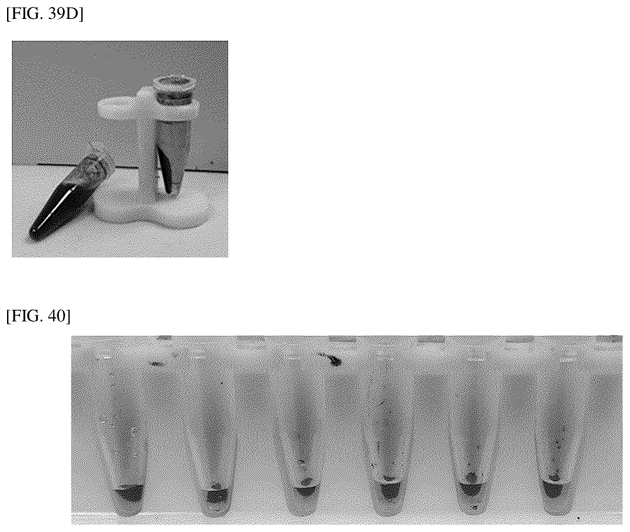

A certain concentration (1-5 mg/ml) of a specific carbon material (e.g., graphene oxide, reduced graphene oxide, graphene, single or multiple wall carbon nanotubes) was mixed with FeCl₂ (0.1-0.6 mg/ml) and FeCl₃ (0.03-0.2 mg/ml) in deionized water. The mixture inserted into an oven was heated up to 85° C. Then, NH₄OH (0.4-0.6%) was added in the mixture. The final mixture was shaken and incubated at 85° C. for an appropriate amount of time (45-60 min) in the oven so that the carbon materials are converted to magnetic carbon nanomaterials. After the incubation, the solution containing magnetic carbon nanomaterials were transferred to a 1.5 ml centrifuge tube. Then, the magnetic carbon nanomaterials were washed 3-4 times with deionized water using a magnetic separator. The pure magnetic carbon materials in deionized water were stored at ambient conditions. As shown in FIGS. 39A-39C the surface of the magnetic carbon nanomaterials (e.g., magnetic graphene oxide containing irons) are different from that of pure graphene oxide. Also, FIG. 39D indicates that antibodies attached on the magnetic graphene oxide can be separated from the solution containing free antibodies remaining after the π-π stacking interaction attaches antibodies to the magnetic graphene oxide.

### 1.2. Preparation of Antibodies Temporarily Immobilized on the Surface of 6 Different Magnetic Carbon Nanomaterials.

Six different types of magnetic carbon nanomaterials were prepared with graphene oxide, reduced graphene oxide, graphene, 10-20 nm, 20-30 nm, and 30-50 nm multi-walled carbon nanotubes based on the method described above. They were stored at ambient conditions.

A specific antibody conjugated with an acridinium ester in PBS was prepared with the addition of acridinium NHS ester capable of binding with primary amine groups of the antibody. The mixture was incubated at room temperature for 30 min. Free antibodies remaining after the reaction were removed by centrifugating it 2-3 times using a centrifugal filtration tube (3, 10, or 30 MWCO). The antibody-conjugated acridinium ester (1 mg/ml) in PBS was stored in a refrigerator.

The antibody-conjugated acridinium ester (1 μg/ml) diluted with the stock solution was mixed with a magnetic carbon nanomaterial (20 μg/ml) in PBS. The mixture, which was inserted into a rotor (18 rpm), was incubated at room temperature for 30 min. After the reaction, the antibody-conjugated acridinium ester immobilized on the surface of magnetic carbon nanomaterial was separated and washed with a magnetic separator. FIG. 40 shows the separations of 6 different magnetic carbon nanomaterials bound with the antibody-conjugated acridinium ester.

1.3. Quantification of D-Dimer with the All-In-One Immunoassay Using Antibodies Attached to the Surface of Magnetic Carbon Nanomaterials.

The antibody-conjugated acridinium ester immobilized on a magnetic carbon nanomaterial (20 μg/ml) in PBS (100 μl) in a 1.5-ml centrifuge tube was inserted into a magnetic separator. Then, clear PBS solution was removed from the 1.5-ml centrifuge tube. Various concentrations of D-dimer in human serum were prepared as standards to operate the all-in-one immunoassay.

A standard containing a certain concentration of D-dimer (100 μl) was added to the 1.5-ml centrifuge tube containing the antibody-conjugated acridinium ester immobilized on a magnetic carbon nanomaterial. The mixture was incubated for 15 min while being rotated (18 rpm) in a rotor.

After the incubation, the solution containing D-dimer antigen-bound antibody-conjugated acridinium ester was taken out from the 1.5-ml centrifuge tube using the magnetic separator.

In order to measure chemiluminescence emitted from the standard, 25 μl of the standard was placed in a borosilicate test tube. Then, the test tube was inserted into the detection cell of the luminometer. After clicking the start button of the luminometer equipped with two syringe injectors, 20 mM in 0.1 N HNO₃ (25 μl) was injected to the test tube through the first syringe pump. After a 3 sec interval, 0.25 M NaOH containing Triton X-100 (125 μl) was injected to the test tube through the second syringe pump to measure chemiluminescence immediately for 2 sec.

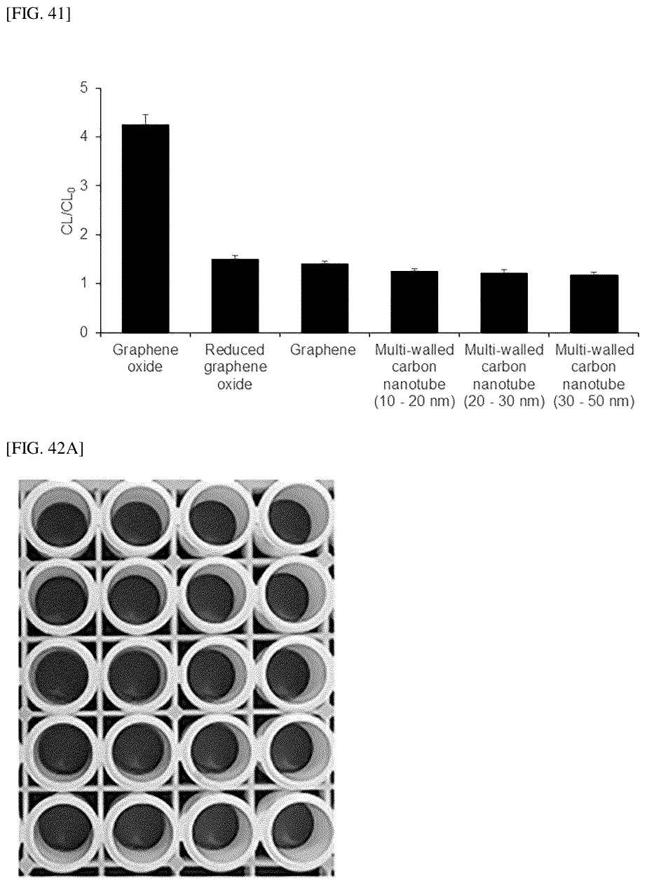

With the increase of D-dimer concentration, relative chemiluminescence intensity was proportionally enhanced. However, the sensitivity of the all-in-one immunoassay was dependent on the properties of magnetic carbon nanomaterials used. For example, FIG. 41 indicates that the all-in-one immunoassay operated with the magnetic graphene oxide is more sensitive than those with other magnetic carbon nanomaterials. This is because the ratio of CL/CL₀ determined with the all-in-one immunoassay with the magnetic graphene oxide is larger than those calculated with other magnetic carbon nanomaterials. CL is the chemiluminescence intensity measured in the presence of 2.5 ng/ml D-dimer, whereas CL₀ is the background measured in the absence of D-dimer. FIG. 39 indicates that the all-in-one immunoassay operated with the magnetic graphene oxide can quantify as low as 2.5 ng/ml D-dimer in human serum with statistically acceptable error range (<5%). The normal range (cut-off value) of D-dimer in human serum is 250 ng/ml in human serum. Thus, the all-in-one immunoassay operated with the magnetic graphene oxide can quantify D-dimer in human serum with good reliability. As shown in FIG. 41, the CL/CL₀ in the presence of other magnetic carbon nanomaterials were slightly higher than 1. These results indicate that the strength of π-π stacking between the antibody and the magnetic graphene oxide is the weakest. Thus, the binding interaction of D-dimer and the antibody occurs on the surface of the magnetic graphene oxide.

### EXAMPLE 2

### All-in-One Immunoassay Operated with Antibodies Temporarily Immobilized on the Surface of Graphene Oxide Film

### 2.1. Produce of Carbon Nanomaterial Film on the Surface of Polystyrene Well

140 μl of a carbon nanomaterial (2 mg/ml) dispersed in aqueous Tris-HCl (10 mM, pH 8.5) was inserted into a polystyrene well of 96-well plate. Six different carbon nanomaterials, such as graphene oxide, reduced graphene oxide, graphene, 10-20 nm multiwalled carbon nanotube, 20-30 nm multiwalled carbon nanotube, and 30-50 nm multiwalled carbon nanotube were used to produce carbon nanomaterial films immobilized on the surface of flat polystyrene well.

Polystyrene wells containing one of 6 different types of carbon nanomaterials were incubated at room temperature or 65 ° C. in an oven to produce carbon nanomaterial films with the evaporation of water.

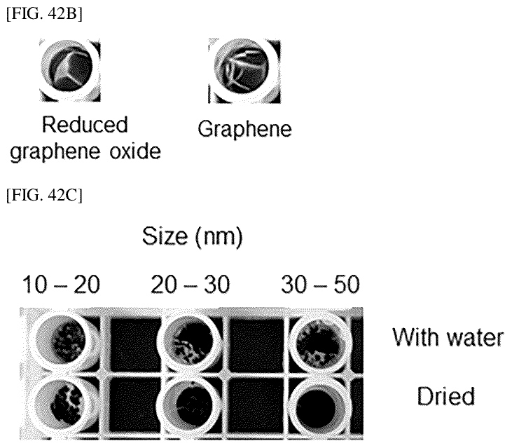

As shown in FIG. 42A, graphene oxide films were produced with the uniform dispersion of graphene oxide particles. The graphene oxide film was stable with the addition of buffer or human serum for at least 24 hours. However, reduced graphene oxide and graphene films on the surface of polystyrene well were split due to the aggregation of nanoparticles as shown in FIG. 42B. The results indicate that it is difficult to apply reduced graphene oxide and graphene films in the all-in-one immunoassay. This is because the precision of the all-in-one immunoassay with split reduced graphene oxide and graphene films is not as good as that with uniform graphene oxide film. Unfortunately, no multi-walled carbon nanotube film was produced. As shown in FIG. 42C, multi-walled carbon nanotubes were dispersed immediately with the addition of PBS even though the dried multi-walled carbon nanotubes on the polystyrene well either look like they are uniform films or have been split.

### 2.2. Graphene Oxide Film Immobilized on the Surface of Polystyrene

A strong graphene oxide film on the surface of polystyrene was produced with the evaporation of water in a buffer solution applied to disperse graphene oxide nanoparticles in a polystyrene well. The production of a uniform graphene oxide film on the surface depends on the components existing in specific buffer solution, pH (6.5-9), and temperature (20-65° C.) in order to evaporate water from the buffer solution.

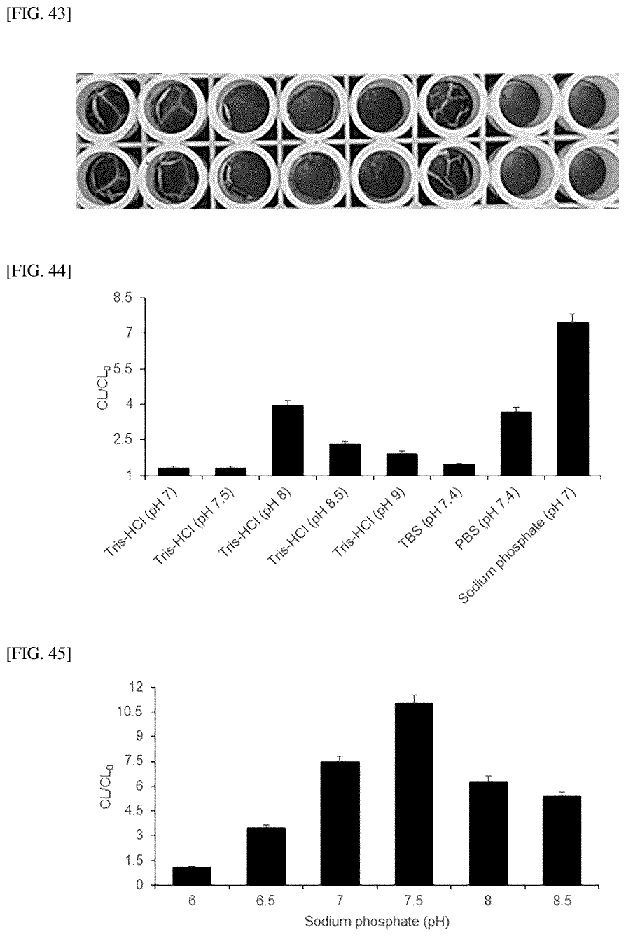

For example, FIG. 43 shows that the uniform graphene oxide films were produced with graphene oxide nanoparticles dispersed in phosphate buffers (pH 7), PBS (pH 7.4), and Tris-HCl (pH 8, 8.5 and 9). However, the graphene oxide films formed in Tris-HCl (pH 7 and 7.5) were irregularly split. The results indicate that the production of strong and uniform graphene oxide films depends on the properties of components, existing in a buffer, mixed with graphene oxide nanoparticles while water in the buffer is evaporated at a certain temperature (20-70° C.). FIG. 43(a) shows the uniform graphene oxide film was formed in PBS (pH 7.4), whereas the irregular and split graphene oxide film was produced in Tris-buffered saline (TBS) (pH 7.4). The uniform graphene oxide films were produced with graphene oxide nanoparticles in phosphate buffer (pH 6.5 or higher) even though the graphene oxide film was split in phosphate buffer (pH 6). The results indicate that major component in producing uniform graphene oxide film in PBS is phosphate ions. NaCl and KCl, components existing in PBS and TBS, aren't a major factor in producing uniform graphene oxide film.

2.3. All-in-One Immunoassay Operated with Graphene Oxide Films Formed in Various Buffers

70 μl of an antibody conjugated with acridinium ester (1 μg/ml) in PBS was added on the surface of graphene oxide films shown in FIG. 43. Then, it was incubated for 30 min at room temperature to temporarily immobilized it on the surface of each graphene oxide film using the π-π stacking interaction between antibodies and a graphene oxide film. After the incubation and washing with PBS, the antibody-conjugated acridinium ester weakly bound on each graphene oxide film was produced.

A standard not containing D-dimer was prepared in order to measure the background (CL₀) in the all-in-one immunoassay. The other standard containing D-dimer (2.5 ng/ml) was prepared to measure chemiluminescence (CL) emitted in the all-in-one immunoassay. Each standard (80 μl) was added in each well containing the antibody-conjugated acridinium ester weakly bound on a graphene oxide film. The mixtures were incubated for 15 min at room temperature. After the incubation, 25 μl of each standard containing D-dimer antigen bound antibody-conjugated acridinium ester or not was added in a borosilicate test tube. The test tube was inserted into the detection cell of luminometer having two syringe pumps. Then, 20 mM H₂O₂ in 0.1 N HNO₃ (25 μl) was injected in the test tube using the first syringe pump. Then, chemiluminescence emitted from each test tube was measured immediately for 2 sec after the addition of 0.25 M NaOH containing Triton X-100 (125 μl) using the second syringe pump.

As shown in FIG. 44, CL/CL₀ was dependent on the property of each graphene oxide film. Also, the results shown in FIG. 44 indicate that the all-in-one immunoassay operated with all the uniform graphene oxide films formed in Tris-HCl (pH 8-9), PBS (pH 7.4) and sodium phosphate (pH 7) can be applied to rapidly quantify trace levels of D-dimer in human serum. The all-in-one immunoassay operated with the graphene oxide film formed in sodium phosphate buffer (pH 7) was more sensitive than other all-in-one immunoassays generated with the graphene oxide films in Tris-HCl buffers (pH 7-9), TBS (pH 7.4), and PBS (pH 7.4). The sensitivities of the all-in-one immunoassays using irregularly split graphene oxide films formed in Tris-HCl (pH 7 and 7.5), and TBS (pH 7.4), as shown in FIG. 43, weren't as good as those using uniform graphene oxide films. Also, the sensitivity of the all-in-one immunoassay operated with a uniform graphene oxide film formed in Tris-HCl buffers is dependent on the pH. As shown in FIG. 45, the all-in-one immunoassay with the uniform graphene oxide film formed in Tris-HCl (pH 8) was more sensitive than those formed in Tris-HCl (pH 8.5 and 9). In addition, the sensitivity of the all-in-one immunoassay operated with a uniform graphene oxide film is dependent on the pH of sodium phosphate buffer. As shown in FIG. 45, the all-in-one immunoassay with the graphene oxide film formed in sodium phosphate buffer (pH 7.5) was more sensitive than the rest of the all-in-one immunoassays with graphene oxide films produced in other sodium phosphate buffers. As shown in FIG. 45, the sensitivity of the all-in-one immunoassay with an irregularly split graphene oxide film formed in sodium phosphate buffer (pH 6) wasn't as good as those with uniform graphene oxide films.

### 2.4 Effect of Buffer Containing Specific Antibodies in the All-in-One Immunoassay

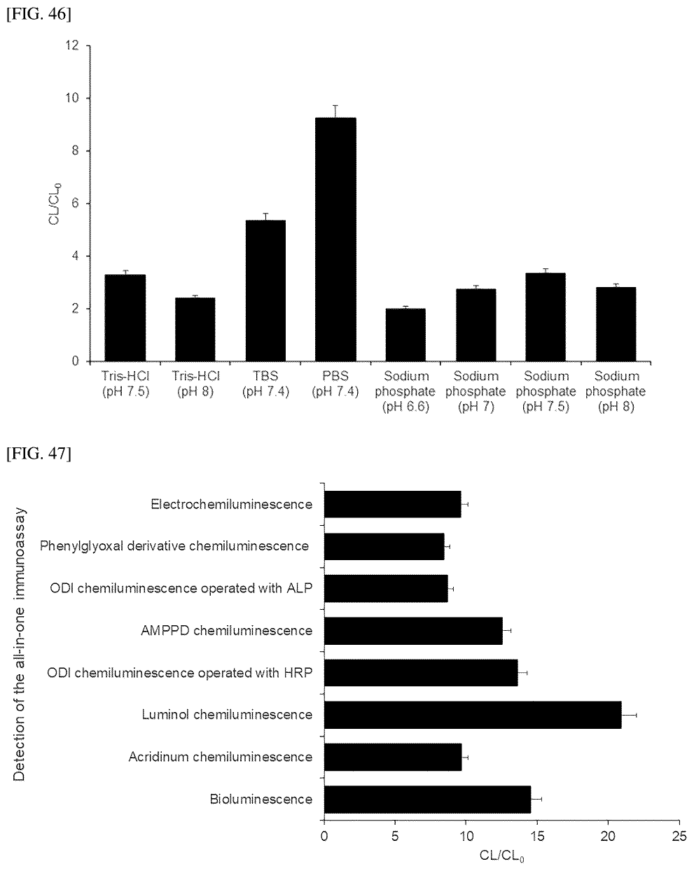

The sensitivity of the all-in-one immunoassay is dependent on buffer containing antibodies capable of weakly binding with a uniform graphene oxide film. The all-in-one immunoassay using PBS (pH 7.4) containing D-dimer antibody conjugated with acridinium ester was more sensitive than the rest of the all-in-one immunoassays using other buffer solutions containing the same antibodies as shown in FIG. 46. Also, the CL/CL₀ of the all-in-one immunoassay using PBS or TBS (pH 7.4) containing D-dimer antibody conjugated with acridinium ester was higher than those using Tris-HCl and sodium phosphate buffers containing the same antibodies. Thus, FIG. 46 indicates that NaCl and KCl can enhance the sensitivity of the all-in-one immunoassay. However, all the all-in-one immunoassays using various buffers containing antibodies and a uniform graphene oxide film can be used to quantify trace levels of D-dimer in human serum.

2.5. CL/CL₀ Determined with the All-in-One Immunoassay with a Bioluminescence, Chemiluminescence, or Electrochemiluminescence Detection

70 μl of the D-dimer antibody conjugated with luciferase (0.25 μg/ml) in PBS (pH 7.4) was added on the surface of graphene oxide film fixed in a polystyrene well. Then, it was incubated for 30 min at room temperature. PBS solution containing the D-dimer antibody conjugated with luciferase remaining after the incubation was removed. Then, the well was washed 3 times with PBS. The D-dimer antibody conjugated with luciferase weakly immobilized on the surface of graphene oxide film was used for the quantification of D-dimer in human serum using the all-in-one enzyme immunoassay with a bioluminescence detection based on the procedure shown in FIG. 7.

70 μl of the D-dimer antibody conjugated with acridinium ester (1 μg/ml) in PBS (pH 7.4) was added on the surface of graphene oxide film fixed in a polystyrene well. Then, it was incubated for 30 min at room temperature. The PBS solution containing the D-dimer antibody conjugated with acridinium ester that remained after the incubation was removed. Then, the well was washed once with PBS. The D-dimer antibody conjugated with acridinium ester weakly immobilized on the surface of graphene oxide film was used for the quantification of D-dimer in human serum using the all-in-one immunoassay with an acridinium chemiluminescence detection based on the procedure shown in FIG. 10A.

70 μl of the D-dimer antibody conjugated with horseradish peroxidase (HRP, 0.1 μg/ml) in PBS (pH 7.4) was added on the surface of graphene oxide film fixed in a polystyrene well. Then, it was incubated for 30 min at room temperature. PBS solution containing the D-dimer antibody conjugated with HRP that remained after the incubation was removed. Then, the well was washed 4 times with PBS. The D-dimer antibody conjugated with HRP weakly immobilized on the surface of graphene oxide film was used for the quantification of D-dimer in human serum using the all-in-one enzyme immunoassay with a luminol detection based on the procedure shown in FIG. 12. Also, the all-in-one enzyme immunoassay with a ODI chemiluminescence detection was operated with the D-dimer antibody conjugated with HRP weakly immobilized on the surface of graphene oxide film for the quantification of D-dimer in human serum, as shown in FIG. 14.

70 μl of the D-dimer antibody conjugated with alkaline phosphatase (ALP, 0.13 μg/ml) in PBS (pH 7.4) was added on the surface of graphene oxide film fixed in a polystyrene well. Then, it was incubated for 30 min at room temperature. PBS solution containing the D-dimer antibody conjugated with ALP that remained after the incubation was removed. Then the well was washed 3 times with PBS. The D-dimer antibody conjugated with ALP weakly immobilized on the surface of graphene oxide film was used for the quantification of D-dimer in human serum using the all-in-one enzyme immunoassay with a AMPPD chemiluminescence detection based on the procedure shown in FIG. 15. Also, the all-in-one enzyme immunoassay with a ODI chemiluminescence detection was operated with the D-dimer antibody conjugated with ALP weakly immobilized on the surface of graphene oxide film for the quantification of D-dimer in human serum, as shown in FIG. 16.

70 μl of the D-dimer antibody conjugated with single stranded DNA-bound 6-FAM (1 μg/ml) in PBS (pH 7.4) was added on the surface of graphene oxide film fixed in a polystyrene well. Then, it was incubated for 30 min at room temperature. PBS solution containing the D-dimer antibody conjugated with single stranded DNA-bound 6-FAM that remained after the incubation was removed. Then, the well was washed once with PBS. The D-dimer antibody conjugated with single stranded DNA-bound 6-FAM weakly immobilized on the surface of graphene oxide film was used for the quantification of D-dimer in human serum using the all-in-one immunoassay with phenylglyoxal derivative detection based on the procedure shown in FIG. 19.

70 μl of the D-dimer antibody conjugated with Ru(bpy)₃²⁺ (1 μg/ml) in PBS (pH 7.4) was added on the surface of graphene oxide film fixed in a polystyrene well. Then, it was incubated for 30 min at room temperature. PBS solution containing the D-dimer antibody conjugated with Ru(bpy)₃²⁺ that remained after the incubation was removed. Then, the well was washed 2 time with PBS. The D-dimer antibody conjugated with Ru(bpy)₃²⁺ weakly immobilized on the surface of graphene oxide film was used for the quantification of D-dimer in human serum using the all-in-one immunoassay with an electrochemiluminescence detection based on the procedure shown in FIG. 32.

The sensitivity of the all-in-one immunoassay was dependent on the properties of material conjugated with D-dimer antibodies, as well as on the detection methods. FIG. 47 shows that every all-in-one immunoassays with a specific detection such as bioluminescence, chemiluminescence, or electrochemiluminescence can rapidly quantify 2.0 ng/ml D-dimer in human serum with good precision. The results indicate that the all-on-one immunoassay with any detection can be applied to rapidly monitor trace levels of D-dimer in human serum.

2.6. Correlation of the All-in-One Immunoassay with a Chemiluminescence Detection

Eight standards containing different troponin I (e.g., 0, 8, 16, 31, 63, 125, 250, 500 pg/ml) in human serum were prepared. They were used to get a linear calibration curve of three all-in-one immunoassays operated with different chemiluminescence detection such as acridinium, luminol, and AMPPD chemiluminescence. Also, 12 unknown human serum samples were used to study the correlation of the 3 different all-in-one immunoassays.

80 μl of a standard (or unknown sample) was placed in a polystyrene well prepared for the all-in-one immunoassay with acridinium chemiluminescence detection. The mixture was incubated for 10 min at room temperature. After the incubation, 25 μl of solution containing (or not containing) TnI antigen-bound antibody-conjugate acridinium ester was transferred to a borosilicate test tube. The test tube was inserted into the detection cell of the luminometer having two syringe pumps. Using the first syringe pump, 20 mM H₂O₂ in 0.1 N HNO₃ (25 μl) was injected into the test tube. After 3 sec, the second syringe pump injected 0.25 M NaOH containing Triton X-100 into the test tube to measure the chemiluminescence intensity for 2 sec. With the increase of TnI concentration, the relative chemiluminescence intensity was proportionally enhanced.

80 μl of a standard (or unknown sample) was placed in a polystyrene well prepared for the all-in-one immunoassay with luminol chemiluminescence detection. The mixture was incubated for 8 min at room temperature. After the incubation, 25 μl of the solution from the polystyrene well was transferred into a borosilicate test tube. The test tube was inserted into the detection cell of the luminometer with two syringe pumps. The first syringe pump injected the mixture (25 μl), containing 7.5 mM H₂O₂ and 1 mM 4-iodophenol, in Tris-HCl (50 mM, pH 8.5) into the test tube. Then, 0.25 mM luminol (25 μl) in Tris-HCl (50 mM, pH 8.5) was injected through the second pump. The final mixture was incubated for 2 min before measuring luminol chemiluminescence emitted from the test tube for 1 sec. With the increase of TnI concentration, the relative chemiluminescence intensity was proportionally enhanced.

80 μl of a standard (or unknown sample) was placed in a polystyrene well prepared for the all-in-one immunoassay with AMPPD chemiluminescence detection. The mixture was incubated for 10 min at room temperature. After the incubation, 10 μl of the solution from the polystyrene well was transferred into a borosilicate test tube. The test tube was inserted into the detection cell of the luminometer with two syringe pumps. The first syringe pump injected the commercially available AMPPD working solution (25 μl) into the test tube. The final mixture was incubated for 3 min before measuring AMPPD chemiluminescence emitted from the test tube for 5 sec. With the increase of TnI concentration, the relative chemiluminescence intensity was proportionally enhanced.

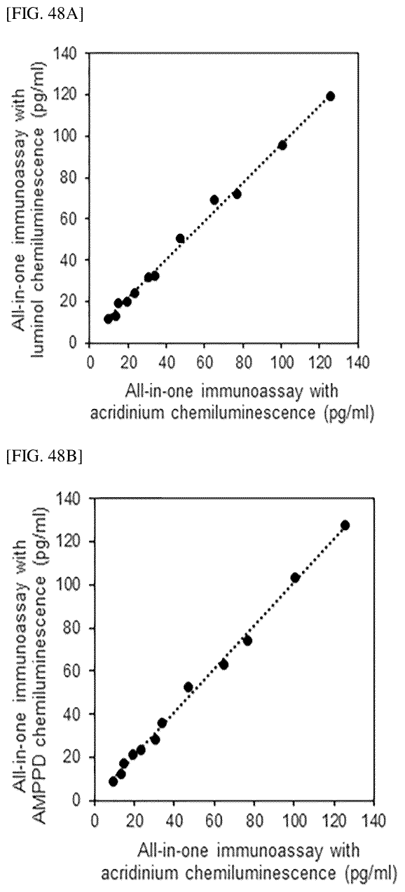

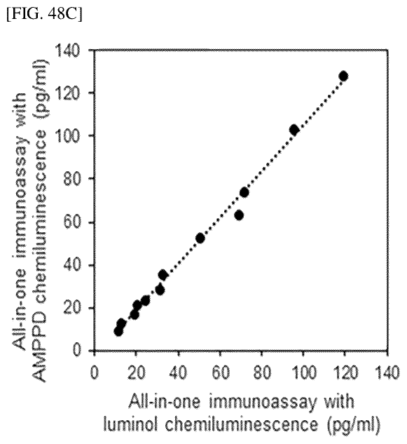

As shown in FIGS. 48A 48B and 48C, the analytical results of unknown samples using the all-in-one immunoassay with a chemiluminescence detection were consistent with those using 2 other chemiluminescence detections. The good correlations shown in FIGS. 48A to 48C indicate that the all-in-one immunoassay can use any chemiluminescence detection for the rapid quantification of TnI in human serum.

### 2.7. Selectivity of the All-in-One Immunoassay

The selectivity of the all-in-one immunoassay is dependent on the specificity of antibody capable of binding a target antigen in a sample. In other words, the selectivity of the all-in-one immunoassay is not determined by a detection method such as bioluminescence, chemiluminescence, colorimetric, electrochemical, electrochemiluminescence, fluorescence, the naked eye, and radiometry. For example, the selectivity of the all-in-one immunoassay with acridinium chemiluminescence designed for the quantification of TnI in human serum was the same as that of other all-in-one immunoassays with a chemiluminescence detection such as luminol, ODI, AMPPD, and phenylglyoxal derivative chemiluminescence.

### C. All-in-One Immunoassay Device (Kit)

The components of the all-in-one immunoassay are determined by (1) a specific antibody conjugated with a material such as bioluminescence emitter (e.g., luciferin derivatives), chemiluminescence emitter (e.g., ABEI, acridinium ester derivatives, luminescent dye, single stranded DNA conjugated with luminescent dye), electrochemiluminescence emitter (e.g., ABEI, isoluminol, luminol, Ru(bpy)₃²⁺), enzyme (e.g., ALP, β-galactosidase, glucose oxidase, HRP, luciferase), nanomaterial (e.g., gold, silver nanoparticles), and radioactive isotope (e.g., ¹²⁵I) and (2) a detection method such as bioluminescence, chemiluminescence, colorimetric, electrochemical, electrochemiluminescence, fluorescence, the naked eye, and radiometry. The all-in-one immunoassay kit has magnetic graphene oxide nanoparticles, or unform graphene oxide film fixed on the surface of polystyrene well capable of weakly capturing a specific antibody-conjugated a biomaterial or chemical material.

While exemplary embodiments of the present disclosure have been described with reference to the accompanying drawings, it will be understood by experts in the art or those of ordinary skill in the art that the present disclosure may be variously modified and changed without departing from the spirit and scope of the present disclosure as defined by the appended claims.

Therefore, the technical scope of the present disclosure should not be limited by the content described in the detailed description of the specification but should be defined by the claims.

What is claimed is:

1. A device for detecting a target antigen in a sample, comprising:
a container;
a carbon nanomaterial; and
an antibody conjugated with a marker,
wherein the antibody is immobilized on a surface of the carbon nanomaterial,
wherein the antibody has a binding site of the target antigen, and
wherein, when the target antigen binds to the binding site of the antibody, the antibody conjugated with the marker detaches from the carbon nanomaterial and the marker-labeled antibody bound with the target antigen generates a signal.

2. The device of claim 1, wherein the carbon nanomaterial is one or more selected from the group consisting of graphene, graphene oxide, reduced graphene oxide, carbon nanotubes, and carbon nanotube oxides.

3. The device of claim 1, wherein the antibody is immobilized on the surface of the carbon nanomaterial by a non-covalent π-π stacking interaction, and a distance between the antibody and the carbon nanomaterial is 10 nm or less.

4. The device of claim 1, wherein the sample is a biological solution selected from the group consisting of serum, plasma, whole blood, sweat, urine, and cerebrospinal fluid.

5. The device of claim 1, wherein the signal generated from the marker is selected from the group consisting of bioluminescence, chemiluminescence, fluorescence, colorimetric, electrochemical, electrochemiluminescence, radiometric, and light visible to the naked eye.

6. The device of claim 1, wherein the marker is a bioluminescence marker selected from the group consisting of luciferase, luciferin, and luciferin derivatives.

7. The device of claim 1, wherein the marker is a chemiluminescence detection marker of a detection selected from the group consisting of acridinium ester chemiluminescence detection, chemiluminescence using horseradish peroxidase (HRP) labeled antibody, chemiluminescence detection using alkaline phosphatase (ALP) labeled antibody, phenylglyoxal derivative chemiluminescence detection, and ODI chemiluminescence detection operated with an antibody conjugated with luminescent dyes.

8. The device of claim 1, wherein the marker is a colorimetric detection marker of a detection selected from the group consisting of colorimetric detection operated with antigen-bound antibody conjugated with horseradish peroxidase (HRP), colorimetric detection operated with antigen-bound antibody conjugated with alkaline phosphatase (ALP), colorimetric detection operated with antigen-bound antibody conjugated with β-galactosidase, and colorimetric detection operated with antigen-bound antibody conjugated with glucose oxidase.

9. The device of claim 1, wherein the marker is an electrochemiluminescence detection marker selected from the group consisting of a ruthenium complex (Ru(bpy)₃²⁺), a gold nanoparticle, a platinum nanoparticle, a silver nanoparticle, and N-(4-aminobutyl)-N-ethylisoluminol (ABEI).

10. The device of claim 1, wherein the marker is an electrochemical detection marker selected from the group consisting of horseradish peroxidase (HRP), alkaline phosphatase (ALP), glucose oxidase, and β-galactosidase.

11. The device of claim 1, wherein the marker is a fluorescence detection marker selected from the group consisting of horseradish peroxidase (HRP), alkaline phosphatase (ALP), and a fluorescence dye.

12. The device of claim 1, wherein the marker is a naked-eye detection marker selected from the group consisting of horseradish peroxidase (HRP) and alkaline phosphatase (ALP).

13. The device of claim 1, wherein the marker is a radioactive detection marker of ¹²⁵I.

14. A method of manufacturing the device of claim 1, comprising:
fixing the carbon nanomaterial on an inner surface of the container;
providing the antibody conjugated with the marker; and
bringing the antibody conjugated with the marker into contact with a surface of the carbon nanomaterial.

15. The method of claim 14, wherein the carbon nanomaterial is a magnetic graphene, graphene oxide, reduced graphene oxide, single-walled carbon nanotube, or multi-walled carbon nanotube.

16. The method of claim 14, wherein the carbon nanomaterial has a form of a thin film composed of graphene oxide, and the container is a polystyrene container.

17. A method of detecting a target antigen, comprising:
introducing a sample solution to the device of claim 1; and
detecting a signal generated from the marker-labeled antibody bound with the target antigen to detect the target antigen in the sample solution.

18. The method of claim 17, wherein the carbon nanomaterial is one or more selected from the group consisting of graphene, graphene oxide, reduced graphene oxide, single-walled carbon nanotubes, and multi-walled carbon nanotubes.

19. The method of claim 17, wherein the carbon nanomaterial has a form of a thin film composed of graphene oxide, and the container is a polystyrene container.

20. The method of claim 17, wherein the antibody is immobilized on the surface of the carbon nanomaterial by a non-covalent π-π stacking interaction, and a distance between the antibody and the carbon nanomaterial is 10 nm or less.

21. The method of claim 17, wherein the sample solution is a biological solution selected from the group consisting of serum, plasma, whole blood, sweat, urine, and cerebrospinal fluid.

22. The method of claim 17, wherein the signal generated from the marker-labeled antibody bound with the target antigen is selected from the group consisting of bioluminescence, chemiluminescence, fluorescence, colorimetric, electrochemical, electrochemiluminescence, radiometric, and light visible to the naked eye.

23. The method of claim 17, wherein the method does not use an artificially manufactured antigen conjugated with the marker.

24. The method of claim 17, wherein the method does not use a detection antibody conjugated with the marker.

25. The method of claim 17, wherein the antibody comprises two or more types of antibodies.

26. The method of claim 17, wherein the method does not comprise a washing procedure to remove waste after introducing the sample solution to the device.

27. The method of claim 17, wherein the signal is emitted within 30 minutes after introducing the sample solution containing the target antigen to the device.

28. A method of quantifying a target antigen, comprising:
introducing a sample solution to the device of claim 1; and
measuring an intensity of the signal to quantify the target antigen in the sample solution.

29. The method of claim 28, wherein the carbon nanomaterial is one or more selected from the group consisting of graphene, graphene oxide, reduced graphene oxide, single-walled carbon nanotubes, and multi-walled carbon nanotubes.

30. The method of claim 28, wherein the antibody is immobilized on the surface of the carbon nanomaterial by a non-covalent π-π stacking interaction, and a distance between the antibody and the carbon nanomaterial is 10 nm or less.

31. The method of claim 28, wherein the sample solution is a biological solution selected from the group consisting of serum, plasma, whole blood, sweat, urine, and cerebrospinal fluid.

32. The method of claim 28, wherein the signal generated from the marker-labeled antibody bound with the target antigen is selected from the group consisting of bioluminescence, chemiluminescence, fluorescence, colorimetric, electrochemical, electrochemiluminescence, radiometric, and light visible to the naked eye.

33. The method of claim 28, wherein the method does not use an artificially manufactured antigen conjugated with the marker.

34. The method of claim 28, wherein the method does not use a detection antibody conjugated with the marker.

35. The method of claim 28, wherein the antibody comprises two or more types of antibodies.

36. The method of claim 28, wherein the method does not comprise a washing procedure to remove waste after introducing the sample solution to the device.

37. The method of claim 28, wherein the intensity of the signal proportionally increases when a concentration of the target antigen in the sample solution increases.

38. The method of claim 28, wherein the signal is generated within 30 minutes after introducing the sample solution to the device.
